(19) United States
(12) Patent Application Publication
(10) Pub. No.: US 2003/0198947 A1
(43) Pub. Date: Oct. 23, 2003

(54) Hepatitis virus sentinel virus I (SVI)

(76) Inventors: Jen-Kuei Liu, Palo Alto, CA (US); Roy A. Bohenzky, Mountain View, CA (US); Yu-Huei Lin, Sunnyvale, CA (US); Benjamin P. Chen, Fremont, CA (US)

Correspondence Address: Roche Diagnostics Corporation, 9115 Hague Road, Bldg. D, Indianapolis, IN, 46250-0457, US

(21) Appl. No.: 10/453,264

(22) Filed: Jun. 3, 2003

Related U.S. Application Data
(63) Continuation of application No. 09/732,665, filed on Dec. 8, 2000, now abandoned.
(60) Provisional application No. 60/172,696, filed on Dec. 10, 1999.

(51) Int. Cl. C12Q 1/70; G01N 33/53; C07H 21/02; C12N 7/00; C12N 15/00; C12N 15/70; C12N 5/16; C07K 1/00; C07K 14/00; C07K 17/00; C12Q 1/68; A61K 39/29; C12N 7/02

(52) U.S. Cl. 435/5; 424/189.1; 435/235.1; 435/239; 435/320.1; 435/326; 435/339; 435/6; 435/7.1; 530/350; 536/23.1; 536/23.72

(57) ABSTRACT

The invention relates to a new group of viruses, designated SVI. Isolated SVI viruses, polynucleotides and proteins from SVI viruses, and antibodies which bind SVI virus and SVI viral proteins are provided. The polynucleotides, proteins, and antibodies of the invention may be used to detect SVI virus or infection by SVI virus in a susceptible individual. Additionally, polynucleotides of the invention may be inserted into recombinant expression vectors recombinant production of viral proteins.

### TECHNICAL FIELD

The invention relates generally to the area of hepatitis viruses, and more particularly to a new group of hepatitis viruses, and methods and compositions for their detection and treatment.

### BACKGROUND

Strictly defined, the term “hepatitis” refers to an inflammation of the liver. A variety of different chemical, viral, and biological agents will induce hepatitis. However, the term hepatitis more commonly refers to an inflammation of the liver caused by a viral infection, particularly a hepatotrophic viral infection.

Viral hepatitis can be divided into two gross categories: acute and chronic. Acute viral hepatitis is characterized by jaundice, malaise, nausea, and elevated blood liver enzymes. Although most cases of viral hepatitis resolve spontaneously, a portion of acute hepatitis victims (generally less than about 10%) develop fulminant necrotizing hepatitis, a disorder with very high morbidity and mortality. Interestingly, many cases of acute hepatitis are so mild as to pass unnoticed or be dismissed as “flu.” Chronic hepatitis gives rise to a much more significant public health problem, and is the most common reason for liver transplant in the United States. Chronic hepatitis is characterized by exacerbations or “flare ups” with symptoms resembling acute hepatitis, as well as portal hypertension and cirrhosis (scarring of the liver) which leads to liver failure. Because acute hepatitis infections can go unnoticed, many chronic hepatitis patients are not diagnosed until their disease is quite advanced, limiting options for treatment.

There are six different families of viruses referred to as “hepatitis viruses” (A, B, C, D, E and G; F having been found to be artifactual). In developed nations, those hepatitis viruses that can establish chronic infections are generally considered to be the most important viruses from a public health standpoint. Of the hepatitis viruses, hepatitis B virus and hepatitis C virus are the only known hepatitis viruses known to establish chronic infections associated with chronic hepatitis. However, HBV and HCV do not account for all cases of transfusion hepatitis. The terms “cryptogenic hepatitis” and “nonA-G” are used to refer to transfusion hepatitis that cannot be attributed to a known hepatitis virus.

Hepatitis B, previously referred to as “transfusion hepatitis” is transmitted via percutaneous, sexual, and vertical routes. The hepatitis B virus, a member of the hepadnaviridae family, can give rise to both acute and chronic hepatitis. The hepatitis B virus (HBV) has been well characterized, and a variety of screening and diagnostic assays are currently available. Additionally, a recombinant vaccine has been created which is currently required for most school age children in the United States.

Hepatitis C, previously known as “non-A, non-B hepatitis” is transmitted primarily via the percutaneous route, although, like HBV, sexual and vertical transmission also occur. Only a minority of acute hepatitis C virus (HCV) infections are clinically apparent, which is problematic because this virus establishes chronic infections at a very high rate. This combination makes chronic HCV infection the leading reason for liver transplant in the United States.

The advent of screening assays for detection of anti-HBV and/or HCV antibodies in donated blood has substantially reduced the transmission of “transfusion hepatitis.” However, 20-30% of infectious blood donations still go undetected. The failure to detect these infectious samples is believed to be largely due to the existence of one or more, as yet unidentified, hepatitis virus.

More recently, in addition to the six known hepatitis viruses, new hepatitis-associated viruses have also been identified. The virus known as TTV was first identified by a Japanese group, who identified genomic sequences from the virus using representation difference analysis (RDA) technology (Nishizawa et al., 1997, Biochem. Biophys. Res. Commun, 241(1):92-97). This virus, which was originally believed to be a member of the parvoviridae family, is a relatively small virus with a buoyant density significantly lower than that of parvoviridae. TTV has been proposed as the prototypic human member of a family of viruses known as circinoviridae for their circular, single-stranded DNA genomes (Mushahwar et al., 1999, Proc. Natl. Acad. Sci. USA, 96(6):3177-3182).

Recently, Diasorin, Inc. has announced the isolation of a new hepatitis virus. The virus, termed SEN-V, is found primarily in blood samples from hepatitis patients, including nonA/nonE or cryptogenic hepatitis patients. Neither the polynucleotide sequence nor methods for isolation of SEN-V have been disclosed.

Accordingly, there is a need in the art for compositions and methods for detection of non-A/non-G hepatitis, as well as compositions and methods for prevention of non-A/non-G hepatitis infections.

### SUMMARY OF THE INVENTION

The invention provides:

1) Compositions comprising isolated SVI virus. Examples of isolated SVI viruses include isolated viruses comprising the polynucleotide sequence of any of SEQ ID NO: 1 through SEQ ID NO: 5.

2) Isolated polynucleotides including an isolated polynucleotide selectively hybridizable with the nucleotide sequence of any of SEQ ID NO: 1 through SEQ ID NO: 5 and complements thereof, an isolated polynucleotide encoding an isolated SVI protein or fragment thereof and complements thereof, wherein said isolated polynucleotide is distinct from the genomic sequences of TTV strains SANBAN and TUS01. The isolated polynucleotide may be an antisense polynucleotide.

3) Compositions comprising an isolated SVI protein or fragment thereof, wherein said isolated SVI protein or fragment thereof is serologically distinct from proteins of TTV strains SANBAN and TUS01.

4) Vaccine compositions comprising an isolated SVI protein or fragment thereof, wherein the isolated SVI protein or fragment thereof is serologically distinct from proteins of TTV strains SANBAN and TUS01. The vaccine compositions may include a pharmaceutically acceptable excipient and/or an adjuvant.

5) Expression vectors comprising an isolated polynucleotide encoding an SVI protein or fragment thereof, wherein said SVI protein or fragment thereof is serologically distinct from proteins of TTV strains SANBAN and TUS01.

6) Expression vectors comprising an isolated polynucleotide, wherein transcription of said isolated polynucleotide results in the production of an SVI antisense polynucleotide, wherein said SVI antisense polynucleotide is not an antisense polynucleotide which will form a duplex with an RNA transcript from TTV strains SANBAN and TUS01.

7) Isolated polyclonal antibodies and monoclonal antibodies which bind to an SVI virus or a protein thereof, wherein said antibody does not bind to TTV strains SANBAN and TUS01 or proteins thereof.

8) Methods for detecting SVI virus, comprising contacting a sample with an antibody which binds to SVI virus or a protein thereof, wherein said antibody does not bind to TTV strains SANBAN and TUS01 or proteins thereof, and detecting complexes of said antibody and SVI virus or protein thereof.

9) Methods for detecting SVI virus, comprising contacting a sample with a probe polynucleotide which selectively hybridizes to an SVI polynucleotide, wherein said probe does not selectively hybridize with TTV strain SANBAN polynucleotides or TTV strain TUS01 polynucleotides; and detecting hybridization of said probe with an SVI polynucleotide.

10) Methods for detecting SVI virus, comprising contacting a sample with a first primer polynucleotide that selectively hybridizes with an SVI polynucleotide and a second primer polynucleotide that hybridizes with a complement of the SVI polynucleotide, performing primer extension DNA synthesis, and detecting the product of the synthesis.

### BRIEF DESCRIPTION OF THE DRAWINGS

FIG. 1 is a graphic depiction of the procedures used in cloning SVI nucleotide sequences.

FIG. 2 summarizes results of assays for serum liver enzymes and SVI in serum samples from chimpanzee X207 inoculated with SVI positive human serum. The arrow indicates the time point of inoculation. Diamonds indicate ALT levels and squares indicate AST levels.

FIG. 3 summarizes the results of assays for SVI in serum from a chimpanzee inoculated with human serum positive for SVI at low titer and SVI positive serum from chimpanzee X207. Arrow 1 indicates the inoculation with human serum positive for SVI at low titer and Arrow 2 indicates inoculation with SVI positive serum from chimpanzee X207. Diamonds indicate prototypic SVI (HFV1) and circles indicate SVI variant.

### DETAILED DISCLOSURE OF THE INVENTION

We have discovered and isolated a new hepatitis virus, designated Sentinel Virus I (SVI), that is associated with cryptogenic, nonA-G hepatitis. The prototypic virus comprises a single stranded DNA genome of at least about 2.6 kilobases. Genomic sequence from the prototypic virus is shown in SEQ ID NO: 1. Accordingly, the invention provides isolated SVI.

We have also discovered that SVI is subject to variability. Accordingly, we have also found members of the SVI family with divergent sequences. The nucleotide sequences of SVI family members with divergent sequences are shown in SEQ ID NO: 1 through SEQ ID NO: 5.

Comparison of the amino acid sequences of the SVI viruses with known virus sequences reveals a low level of homology with certain variants of the circinoviridae family.

In one aspect, the invention provides isolated polynucleotides comprising the SVI viral genome and fragments thereof. The polynucleotides may be DNA or RNA. Also provided are isolated nucleotide probes or primers for use in detecting SVI infections and/or SVI virus itself. The probes and/or primers may also be used in methods for identification and isolation of new variants of SVI.

A further aspect of the invention provides isolated SVI viral proteins and/or fragments thereof, as well as fusion proteins comprising an SVI viral protein or fragment thereof fused with a heterologous (non-SVI) protein. Also included are mosaic polypeptides that comprise at least two SVI epitopes. In mosaic polypeptides of the invention comprising two epitopes from the same SVI protein, the intervening amino acids between the epitopes are substantially deleted or substituted with a heterologous sequence. Alternately, mosaic polypeptides of the invention may comprise two epitopes from different SVI proteins or comprise homologous epitopes from at least two viruses of the SVI family.

The invention provides recombinant expression constructs, comprising a polynucleotide sequence derived from an open reading frame of an SVI virus operably linked to promoter operable in a prokaryotic or eukaryotic host cell. Also provided are expression vectors and recombinant host cells comprising the expression constructs.

Also provided are antibodies specific for epitopes of the SVI family of viruses. Included are monoclonal antibodies and isolated polyclonal antibodies.

In another aspect, the invention provides assays and kits for conducting assays for detection of SVI infection and/or detection of SVI virus. The assays of the invention may be immunoassays utilizing polypeptides or antibodies of the invention or nucleic acid-based assays employing hybridization or amplification technology with one or more polynucleotides of the invention.

In a further aspect the invention provides vaccines for prevention and/or treatment of SVI infection. The vaccines comprise one or more polypeptides derived from SVI, optionally combined with an adjuvant.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987 and periodic updates); “PCR: The Polymerase Chain Reaction”, (Mullis et al., eds., 1994); “Current Protocols in Immunology” (J. E. Coligan et al., eds., 1991); and “Immunochemistry in Practice” (Johnstone and Thorpe, eds., 1996; Blackwell Science).

Definitions

The terms “Sentinel Virus I” and “SVI” refer to a virus, type of virus, or class of virus which is transmissible via percutaneous exposure in humans and is serologically distinct from hepatitis A virus (HAV), hepatitis B virus (HBV), hepatitis C virus (HCV), hepatitis D virus (HDV), hepatitis E virus (HEV), hepatitis G virus (HGV), and TTV variants SANBAN and TUS01. SVI comprises a genome with a major open reading frame (ORF) with at least about 40%, 50%, 55%, 60%, 65%, 70%, 75%, 80%, 85%, 90%, 95%, 97%, 98%, 99% or 100% global amino acid sequence homology with the amino acid sequence of SEQ I. NO: 6 and/or at least about 40%, 50%, 55%, 60%, 65%, 70%, 75%, 80%, 85%, 90%, 95%, 97%, 98%, 99% or 100% global amino acid sequence identity with the amino acid sequence of SEQ ID NO: 6. Alternately, an SVI variant may have at least about 40%, 50%, 55%, 60%, 65%, 70%, 75%, 80%, 85%, 90%, 95%, 97%, 98%, 99% or 100% global nucleotide sequence identity with the sequence of SEQ ID NO: 1, encode an ORF with at least about 40%, 50%, 55%, 60%, 65%, 70%, 75%, 80%, 85%, 90%, 95%, 97%, 98%, 99% or 100% global amino acid sequence homology with the amino acid sequence of SEQ ID NO: 6 and/or at least about 40%, 50%, 55%, 60%, 65%, 70%, 75%, 80%, 85%, 90%, 95%, 97%, 98%, 99% or 100% global amino acid sequence identity with the amino acid sequence of SEQ ID NO: 6. The term SVI also refers to the prototypic SVI and naturally occurring variants of SVI.

An “SVI polypeptide” or “SVI protein” is a polypeptide or protein encoded by an ORF of an SVI virus genome which is not encoded by a known virus ORF such as an ORF of a known member of the circinoviridae family, for example TTV variants SANBAN and TUS01, the sequences of which are publicly available through the Genbank database under accession numbers AB025946 and AB017613, respectively. Exemplary SVI polypeptides are shown in the amino acid sequences shown in SEQ ID NO: 6 through SEQ ID NO: 12. Preferably, an SVI polypeptide is at least about 8, 10, 12, 15, 20, 25, 30, 40, or 50 amino acids and less than about 800, 700, 500, 400, 300, 250, 200, 150, 125, or 100 amino acids.

A “variant SVI polypeptide” is a polypeptide which has at least about 40%, 50%, 55%, 60%, 65%, 70%, 75%, 80%, 85%, 90%, 95%, 97%, 98%, 99% or 100% amino acid sequence homology with the corresponding amino acid sequence of any of SEQ ID NO: 6 through SEQ ID NO: 12 and/or at least about 40%, 50%, 55%, 60%, 65%, 70%, 75%, 80%, 85%, 90%, 95%, 97%, 98%, 99% or 100% amino acid sequence identity with the corresponding portion of any of the amino acid sequence of SEQ ID NO: 6 through SEQ ID NO: 12. Preferably, a variant SVI polypeptide is at least about 8, 10, 12, 15, 20, 25, 30, 40, or 50 amino acids and less than about 800, 700, 500, 400, 300, 250, 200, 150, 125, or 100 amino acids.

A “SVI polynucleotide” is a polynucleotide with a sequence identical to a polynucleotide or fragment thereof shown in any of SEQ ID NO: 1 through SEQ ID NO: 5, a complement thereof, or a polynucleotide which encodes an SVI polypeptide or the complement thereof. An SVI polynucleotide is not found in any known sequence, particularly in a known variant of the circinoviridae, for example TTV variants SANBAN and TUS01. Preferably, an SVI polynucleotide is at least about 15, 20, 25, 30, 35, 40, 50 or 60 nucleotides and less than about 2500, 2000, 1500, 1250, 1000, 900, 800, 700, 600, 500, 400, 300, 200, 150, 125, 100, 75 or 50 nucleotides in length. A “complement” to a polynucleotide of interest is a polynucleotide which is capable of hybridizing under moderate or high stringency conditions, using Watson/Crick base pairing, to the polynucleotide of interest.

A “variant SVI polynucleotide” is a polynucleotide which encodes a variant SVI polypeptide or complement thereof or a polynucleotide which is selectively hybridizable to an SVI polynucleotide or complement thereof, but does not fall within the definition of an SVI polynucleotide. A variant SVI polynucleotide is not found in any known sequence, particularly in a known variant of the circinoviridae family. Preferably, a variant SVI polynucleotide is at least about 15, 20, 25, 30, 35, 40, 50 or 60 nucleotides and less than about 2500, 2000, 1500, 1250, 1000, 900, 800, 700, 600, 500, 400, 300, 200, 150, 125, 100, 75 or 50 nucleotides in length.

“Amino acid sequence homology” and “amino acid sequence identity” refer to the percentage of amino acids that are homologous or the same in comparing the two sequences. This alignment and the percent sequence homology or sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. For purposes of the present invention, the alignment program is BLASTP, using the following default parameters: databases=non-redundant (non-redundant GenBank CDS translations+PDB+SwissProt+PIR+PRF), low complexity filtering=ON, expect=10, matrix=BLOSUM62 (gap existence cost 11, gap per residue 1, lambda 0.85) and word size=3. Alignment may be performed gapped or ungapped, and is preferably performed gapped. Details of this BLASTP implementation and these parameters can be found at the following Internet address: http://www.ncbi.nlm.nih.gov/cgi-bin/BLAST.

“Nucleotide sequence identity” refers to the percentage of nucleotide residues which are the same in comparing the two sequences. This alignment and the percent sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. For purposes of the present invention, the alignment program is BLASTN, using the following default parameters: databases=non-redundant (all non-redundant GenBank+EMBL+DDBJ+PDB sequences), low complexity filtering=ON, expect=10, matrix=BLOSUM62, gap existence cost=5, gap extension cost=2, mismatch penalty=−3, match reward=1, and word size=11. Alignment may be performed gapped or ungapped, and is preferably performed gapped. Details of this BLASTN implementation and these parameters can be found at the following Internet address: http://www.ncbi.nlm.nih.gov/cgi-bin/BLAST.

A polynucleotide which is “selectively hybridizable” to an SVI polynucleotide sequence is one which (i) hybridizes to an SVI polynucleotide sequence without hybridizing to a known virus polynucleotide sequence such as sequence from one of the known members of the circinoviridae or specifically primes amplification of an SVI polynucleotide sequence without priming amplification of a known virus polynucleotide sequence such as sequence from one of the known members of the circinoviridae. Hybridization of a selectively hybridizable polynucleotide may be accomplished at high stringency, moderate stringency, or low stringency (e.g., allowing for mismatches), as appropriate. High stringency conditions utilize a final wash that is 12-20° C. below the Tₘ of the expected hybrid, while moderate and low stringency hybridizations utilize final wash conditions which are 21-30° C. and 31-40° below the Tₘ of the hybrid. The Tₘ of a long polynucleotides can be found as Tₘ=81.5−16.6(log₁₀[Na⁺])+0.41(%G+C)−0.63(%formamide)−600/N, where N=the length of the selectively hybridizable polynucleotide under study, while the Tₘ of oligonucleotides from about 70 to 15 nucleotides in length may be found as Tₘ=81.5−16.6(log10[Na+])+0.41(%G+C)−600/N, and the Tₘ of short oligonucleotides of ≦14 nucleotides may be found as Tₘ=2(A+T)+4(G+C). Priming of amplification is preferably carried out under standard conditions for the polymerase chain reaction (PCR) (e.g., 50 mM KCl, 10 mM Tris-HCl, pH 8.3 (at 20° C.), 1.5 mM MgCl₂, optionally with 0.01% gelatin) and T. aquaticus DNA polymerase.

An “isolated” virus, viral structure (e.g., capsid), polynucleotide or polypeptide is one that has been at least partially purified away from contaminating components found in its normal environment. For example, an isolated virus is one that has been at least partially purified away from blood, serum, or tissue proteins. In the case of an isolated viral polynucleotide, the polynucleotide is at least partially purified away from viral proteins and/or other viral components and may additionally be removed from its normal milieu (e.g., nucleotide sequences which normally flank the polynucleotide may be deleted).

As used herein, a sequence of interest and regulatory sequences are said to be “operably linked” when they are covalently linked in such a way as to place the expression or transcription of the sequence of interest under the influence or control of the regulatory sequences. The term “operably linked” relates to the orientation of polynucleotide elements in a functional relationship. Operably linked means that the DNA sequences being linked are generally physically contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous. If it is desired that a sequence of interest be translated into a functional protein, two DNA sequences are said to be operably linked if induction of a promoter in the 5′ regulatory sequences results in the transcription of the sequence of interest and if the nature of the linkage between the two DNA sequences does not (1) result in the introduction of a frame-shift mutation, (2) interfere with the ability of the promoter region to direct the transcription of the sequence of interest, or (3) interfere with the ability of the corresponding RNA transcript to be translated into a protein. Thus, a promoter region would be operably linked to a sequence of interest if the promoter region were capable of effecting transcription of that DNA sequence such that the resulting transcript might be translated into the desired protein or polypeptide.

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only intact antibody molecules but also fragments of antibody molecules retaining antigen binding ability. Such fragments are also well known in the art and are regularly employed both in vitro and in vivo. In particular, as used herein, the term “antibody” means not only intact immunoglobulin molecules of any isotype (IgA, IgG, IgE, IgD, IgM) but also the well-known active (i.e., antigen-binding) fragments F(ab′)₂, Fab, Fv, scFv, Fd, V_{H} and V_{L}. For antibody fragments, see, for example “Immunochemistry in Practice” (Johnstone and Thorpe, eds., 1996; Blackwell Science), p. 69. The term “antibody” further includes single chain antibodies, CDR-grafted antibodies, diabodies, chimeric antibodies, humanized antibodies, and a Fab expression library. The term also includes fusion polypeptides comprising an antibody of the invention and another polypeptide or a portion of a polypeptide (a “fusion partner”). Examples of fusion partners include biological response modifiers, lymphokines, cytokines, and cell surface antigens. “Antibody activity” refers to the ability of an antibody to bind a specific antigen in preference to other potential antigens via the antigen combining site located within a variable region of an immunoglobulin. As used herein, the term “serologically distinct” describes a polypeptide, protein or virus that can be immunologically identified by specific antibodies as distinct from other species of polypeptides, proteins or viruses by virtue of its antigenic differences from such other species.

As used herein, the term “comprising” and its cognates are used in their inclusive sense; that is, equivalent to the term “including” and its corresponding cognates.

Isolated SVI Virus

Isolated SVI is preferably prepared from plasma or serum derived from an SVI infected individual. SVI virus may be isolated from serum or plasma using any technique known in the art, including, but not limited to, isopycnic gradient centrifugation, particularly at preparative scale, and immunoisolation. Isopycnic gradient centrifugation may be performed using any gradient-forming compound known in the art that will form a gradient in the range of 1.20 to 1.40 g/mL; sucrose and cesium chloride (CsCl) are preferred gradient forming compounds. Plasma or serum containing SVI is ‘layered’ over the gradient forming compound (which may be in a preformed gradient or homogenous, depending on the gradient forming compound) in an appropriate centrifuge tube, then centrifuged to equilibrium. Isolated SVI virus may be recovered by collecting the appropriate density fraction of the gradient. For example, where the gradient forming compound is CsCl, the 1.33-1.35 g/cm³ fraction is collected. Immunoisolation techniques utilize SVI virus-specific antibodies in combination with any appropriate separation media known in the art. Preferred separation media include solid plastic substrates (e.g., as for use in panning), chromatographic media (e.g., immunoaffinity chromatography) and magnetic particles (e.g., immunomagnetic separation). The anti-SVI antibodies are conjugated to the separation media, which is exposed to a material containing SVI virus. Unbound materials are removed and residual unbound materials are washed away from the immunoseparation substrate, then the bound SVI virus is eluted, typically by use of an elution buffer with altered pH or high salt concentration.

Alternately, isolated SVI virus may be prepared by in vitro culture methods.

Isolated SVI Polynucleotides

Isolated SVI polynucleotides may be prepared by any method known in the art, such as by direct isolation of viral DNA from viral particles, by direct isolation of viral RNA transcribed as part of the SVI life cycle, by use of a hybridization method (i.e., identification of viral DNA in DNA libraries prepared from viral DNA, or virus-containing serum or plasma), by use of an amplification method (i.e., polymerase chain reaction of viral DNA, viral-DNA containing libraries, or DNA isolated from plasma or serum), or by direct synthesis. The polynucleotide sequences shown in SEQ ID NO: 1 through SEQ ID NO: 5 may be used to design probes or primers for use in hybridization and amplification methods and to select sequences for synthesis.

Isolated genomic polynucleotides may be prepared by extraction of isolated viral particles. Isolated viral particles may be subjected to any DNA extraction technique known in the art, such as guanidinium HCl extraction, optionally followed by further purification and/or concentration techniques such as agarose gel purification, phenol/chloroform extraction, or ethanol precipitation in the presence of salts.

Preparation of DNA libraries is well known in the art. DNA isolated from viral particles or virus-containing plasma or serum may be cloned into a convenient library vector using techniques commonly used in the art. Most commonly, the library will be prepared using a lambda phage-based library vector, although cosmid and plasmid libraries are also commonly used. Phage-based libraries are plated by infection of ‘lawns’ of E. coli host cells, while cosmid and plasmid libraries are typically transformed into cells which are plated. After plating, DNA from the library is transferred to screening filters, and screening with an SVI polynucleotide probe. The probe is preferably modified such that hybridization can be detected, typically by the incorporation of a radioactive nucleotide (e.g., ³²P), although other modified probes (e.g., digoxigenin or biotin labeled) may be detected through the use of a modified enzyme (e.g., alkaline phosphatase or luciferase) which binds the labeled probe and acts on a chromogenic or otherwise detectable substrate. Clones hybridizing to the SVI polynucleotide probe are purified by one or more ‘rounds’ of purification (e.g., repeating the plating and screening process on progressively more purified clones), as is well known in the art. SVI virus DNA may be prepared by harvesting DNA from clone isolated in the screening procedure, and optionally further isolated from the library vector DNA by restriction endonuclease digestion. Alternately, clone DNA isolated by screening may be used as a substrate for amplification of SVI virus DNA using polymerase chain reaction (PCR) methodology. PCR primers may be designed from SVI virus DNA or, more conveniently, may be designed to hybridize to DNA sequences in the library vector which flank the site at which the library DNA was inserted, as will be apparent to one of skill in the art.

SVI virus polynucleotides may also be isolated by amplification from samples containing SVI virus DNA. Primers for amplification may be designed based on the sequences shown in SEQ ID NO: 1 through SEQ ID NO: 5, and are preferably designed so as to amplify SVI DNA, but not viral DNA from TTV or TTV variants. Additionally, as is well known in the art, the primer sequences are selected to minimize any intramolecular secondary structure, which substantially inhibits, and may even block, amplification. Protocols for polymerase chain reaction amplification are well known in the art, as are protocols for other amplification methods such as ligation chain reaction. After amplification, the SVI DNA may be further purified by size selection (e.g., gel electrophoresis) or chemical extraction (e.g., phenol/chloroform extraction) and/or concentrated by ethanol precipitation in the presence of salts.

SVI polynucleotides may also be chemically synthesized, although synthesized SVI polynucleotides are preferably less than about 50-60 nucleotides in length, as yields for polynucleotide synthesis drop as chain length increases. Methods for synthesis of polynucleotides are well known in the art, and generally involve the iterative addition of nucleotides (or modified nucleotides) to the growing end of the synthetic polynucleotide. A variety of different systems are available in the art, and the selection of the particular method and chemistry is left to the practitioner.

SVI polynucleotides have a variety of uses, including detection of SVI virus (which is useful in diagnosis of SVI infection), production of SVI polypeptides, construction of SVI-based expression/transduction vectors, and as antisense oligonucleotides or for construction of antisense SVI vectors.

Antisense SVI polynucleotides are SVI polynucleotides which are capable of selective hybridization to a segment of an mRNA molecule produced from an SVI genome. Antisense SVI polynucleotides may be any size SVI polynucleotide, but are preferably less than about 200 nucleotides in length. Antisense SVI polynucleotides block expression of SVI proteins and/or SVI viral replication in SVI infected cells. Accordingly, SVI antisense polynucleotides may be used to treat SVI infections and/or ameliorate the symptoms of SVI infections, including reduction of SVI viremia.

When SVI antisense polynucleotides are chemically synthesized, they are preferably synthesized as modified oligonucleotides to increase resistance to nucleases. Modified oligonucleotides may be synthesized to include phosphoroamidites at the 5′ and 3′ termini (Dagle et al., 1990, Nucl. Acids Res. 18:4751-4757), to incorporate the ethyl or methyl phosphonate analogs disclosed in U.S. Pat. No. 4,469,863, to incorporate phosphorothioate (Stein et al., 1988. Nucl. Acids Res. 16:3209-3221) or 2′-O-methylribonucleotides (Inove et al., 1987, Nucl. Acids Res. 15:6131), or as chimeric oligonucleotides that are composite RNA-DNA analogues (Inove et al., 1987, FEBS Lett. 215:327).

SVI antisense polynucleotides may be delivered to individuals infected with SVI virus as “naked DNA,” normally by parenteral injection, preferably by intravenous or introduction into the portal vein, exploiting the naturally occurring uptake of oligonucleotides. Alternately, SVI antisense polynucleotides may be introduced into target cells via a vector, such as a viral vector. The vector comprises a promoter operable in a host cell, preferably a human host cell infected with SVI, operably linked to a polynucleotide sequence, transcription of which results in the production of an SVI antisense polynucleotide. Preferred viral vectors include, but are not limited to, the adeno-associated viral vectors known in the art. Preferably, SVI antisense polynucleotides delivered by viral vector are administered intravenously, preferably into the portal vein.

Isolated SVI Polypeptides

SVI proteins may comprise an entire ORF from an SVI virus, one or more fused proteins from an SVI virus, a single protein from an SVI virus, or fragments thereof. Also included are “mosaic proteins” which comprise two or more SVI protein fragments within the same protein. The SVI protein fragments in a mosaic protein may be from the same SVI protein or from different SVI proteins. Where the SVI protein fragments are from the same SVI protein, the amino acid sequence normally separating the fragments is substantially deleted or replaced with an unrelated “spacer” sequence. Another mosaic protein encompassed by the invention is a “superepitope” mosaic protein that comprises homologous versions of at least one epitope from at least two different SVI viruses. Superepitope mosaic proteins may be used, for example, in screening assays to generically detect SVI virus infection.

SVI polypeptides may be prepared by any method known in the art, including purification from isolated viral particles, recombinant production and chemical synthesis. Due to the relative difficulty of isolating large amounts of viral particles from natural sources and the variability in the SVI virus family, recombinant production and/or chemical synthesis are preferred methods for production.

Recombinant production of proteins is well known in the art. Generally, a polynucleotide sequence encoding a protein of the invention is cloned into an “expression construct,” which is introduced into a suitable host cell. The host cell is cultured under conditions appropriate for expression of the protein, and the recombinant protein is collected. The exact details of the expression construct will, as will be apparent to one of skill in the art, vary depending on the desired host cell and properties of the expression construct, although the expression construct will normally include a promoter/operator or promoter/enhancer operable in the host cell and a selectable marker allowing selection of cells containing the marker. Preferably the promoter/operator or promoter/enhancer is ‘controllable’ in that a change in culture conditions will lead to expression of the SVI protein (or SVI protein fusion protein).

It should be noted that SVI peptides may be incorporated into “fusion proteins” for recombinant production. A fusion protein comprises a protein of interest (e.g., the SVI protein) linked to a fusion partner, and optionally includes a specific cleavage site between the protein of interest and the fusion partner to allow separation of the two parts. The fusion partner may be at the amino terminal or carboxy terminal of the protein, although fusion proteins which incorporate the protein of interest as an ‘insert’ within the sequence of the coding region are also contemplated. Fusion proteins comprising an SVI protein insert may be particularly useful as screening tools, for example, when incorporated into a “phage display” system (e.g., where the SVI protein sequence is inserted into a lambda phage coat protein).

Useful fusion partners include proteins which allow for easy purification of the fusion protein (e.g., glutathione-S-transferase, oligo-histidine, and certain sequences derived from the myc oncogene), increase solubility of the fusion protein (such as E. coli DsbA, disclosed in U.S. Pat. No. 5,629,172), or create a “linker” to bind the protein to a substrate (e.g., polyglycine with a terminal lysine could be used to link a c 37 protein to substrate for use in an immunoassay).

Generally, an expression construct is created by inserting a polynucleotide encoding a protein of the invention into an appropriate recombinant DNA expression vector using appropriate restriction endonucleases. The restriction endonuclease sites may be naturally occurring or synthetic sites that have been introduced by any method known in the art, such as site-directed mutagenesis, PCR or ligation of linker/adapters to the polynucleotide. Alternately, the polynucleotide may be a synthetic sequence, designed to incorporate convenient restriction enzyme sites and/or optimize codon usage for the intended host cell. The particular endonucleases employed will be dictated by the restriction endonuclease cleavage pattern of the parent expression vector to be employed. The choice of restriction sites is made so as to properly orient the coding sequence with control sequences to achieve proper in-frame reading and expression of the protein.

The polynucleotide may be inserted into any appropriate expression vector. Expression vectors may be found in a number of forms, including, but not limited to, plasmid, cosmid, yeast artificial chromosome (YAC), and viral. In general, the expression vector will contain an autonomous replication site that is active at least in the organism in which the vector is propagated, and frequently also in the recombinant host cell. The expression vector will also typically include marker sequences which are capable of providing phenotypic selection in transformed cells, such as positive selection markers, such as an antibiotic resistance genes (e.g., bla, tet^{R}, neo^{R} or hyg^{R}) or genes which complement an auxotrophy (e.g., trp or DHFR) and/or negative selection markers such as herpes simplex virus 1 thymidine kinase. The expression vector will also include necessary sequences for initiation and termination of transcription and translation (e.g., promoter, Shine-Dalgarno sequence, ribosome binding site, transcription termination site) and may optionally contain sequences which modulate transcription (e.g., SV40 enhancer or lac repressor), and may also contain sequences which direct processing, such as an intron or a polyadenylation site, as necessary.

The polynucleotide of the invention is inserted into the expression vector in the proper orientation and relationship with the expression vector's transcriptional and translational control sequences to allow transcription from the promoter and translation from the ribosome binding site, both of which should be functional in the host cell in which the protein is to be expressed. The transcriptional control sequences are preferably inducible (i.e., can be modulated by altering the culture conditions, such as the lac operon for E. coli or the metallothionein promoter for mammalian cells). An example of such an expression vector is a plasmid described in Belagaje et al., U.S. Pat. No. 5,304,493. The gene encoding A-C-B proinsulin described in the reference can be removed from the plasmid pRB182 with restriction enzymes NdeI and BamHI. The genes encoding the protein of the present invention can be inserted into the plasmid backbone on a NdeI/BamHI restriction fragment cassette.

Microbial hosts are normally preferred for recombinant expression of the proteins of the invention, and any commonly used microbial host, including E. coli such as W3110 (prototrophic, ATCC NO. 27325), Bacillus subtilis, and other enterobacteriaceae such as Salmonella typhimurium or Serratia marcescans, and various pseudomonas species may be used. Alternately, eukaryotic host cells, including yeast such as Saccharomyces cerevisiae, Schizosaccharomyces pombe, as well as higher eukaryotes such as non-yeast fungal cells, plant cells, insect cells (e.g., Sf9), and mammalian cells (e.g., COS, CHO) may be used.

The completed expression construct is introduced into the recombinant host cell by any appropriate method known in the art, such as CaCl₂ transfection, Ca₂PO₄ transfection, viral transduction, lipid-mediated transfection, electroporation, ballistic transfection, and the like. After introduction of the expression construct, the recombinant host cell is generally cultured under appropriate conditions to select for the presence of the expression construct (e.g., cultured in the presence of ampicillin for a bacterial host with an expression construct containing bla), or alternately may be selected for expression of the protein by any appropriate means (e.g., fluorescence activated cell sorting, FACS, using an SVI protein-specific antibody).

After selection and appropriate isolation procedures (e.g., restreaking or limiting dilution cloning), the recombinant host cells are cultured at production scale (which may be from 500 mL shaken flask to multi-hundred liter fermenter for microbial host cells, or from T25 flask up to multi-hundred liter bioreactor for mammalian host cells, depending on the requirements of the practitioner), using any appropriate technology known in the art. If the promoter/enhancer in the expression vector is inducible, the expression of the protein is induced as appropriate for the particular construct (e.g., by adding an inducer, or by allowing a repressor to be depleted from the media) after the culture reaches an appropriate cell density, otherwise the cells are grown until they reach appropriate density for harvest. Harvesting of the recombinant proteins of the invention will depend on the exact nature of the recombinant host cells, the expression construct, and the polynucleotide encoding the protein of the invention, as will be apparent to one of skill in the art. For expression constructs that result in a secreted protein, the protein is normally recovered by removing media from the culture vessel, while expression constructs that result in intracellular accumulation of the protein generally require recovery and lysis of the cells to free the expressed protein.

Proteins which are expressed in high-level bacterial expression systems characteristically aggregate in granules or inclusion bodies which contain high levels of the overexpressed protein. The protein aggregates are solubilized to provide further purification and isolation of the desired protein product, for example, using strongly denaturing solutions such as guanidinium-HCl, possibly in combination with a reducing agent such as dithiothreitol (DTT). The solubilized protein is recovered in its active form after a “refolding” reaction, in which generally involves reducing the concentration of the denaturant and adding oxidizing agent. Protocols which are considered generally applicable for the refolding of proteins are well known in the art, and are disclosed in, for example, U.S. Pat. Nos. 4,511,502, 4,511,503, and 4,512,922.

Short (e.g., less than about 20 amino acid residues) SVI proteins may also be conveniently produced using synthetic chemistry, a process well known in the art. Due to decreased yields at long peptide lengths, synthesis is a preferred method for production of peptides of about 15 amino acid residues or less.

SVI polypeptides may be used in vaccines for prevention of SVI infection and/or treatment of SVI infection. Any SVI polypeptide or combination of SVI polypeptides maybe used in an SVI vaccine. SVI mosaic polypeptides comprising multiple epitopes from a single SVI protein, wherein the amino acids normally separating the epitopes are deleted are one preferred SVI protein for use in a vaccine formulation. Another preferred SVI protein for use in a vaccine is a superepitope protein that comprises homologous epitopes from multiple SVI viruses fused into a single protein.

SVI vaccines are formulated according to methods known in the art. Preferably the vaccine is in a liquid formulation for parenteral administration. The vaccines may be formulated including pharmaceutical excipients known in the art such as physiologically and pharmaceutically acceptable salts, buffers, preservatives, bulking agents, osmolyte agents, and the like, which may be found in the USP (United States Pharmacopeia, United States Pharmacopeial Convention, Inc., Rockville, Md., 1995).

SVI protein-based vaccines may also be formulated with adjuvants. Adjuvants for use in SVI-protein based vaccines include chemical adjuvants such as aluminum hydroxide (especially aluminum hydroxide gels), potassium alum, protamine, aluminum phosphate and calcium phosphate, cytokine adjuvants including interleukin (IL)1_{β}, tumor necrosis factor (TNF)_{α} and granulocyte-macrophage colony stimulating factor (GM-CSF), such as described in U.S. Pat. No. 5,980,911, and oil in water emulsions such as Freund's complete and incomplete adjuvants.

SVI vaccines are preferably delivered parenterally, more preferably by percutaneous administration. Prefered routes of administration include intramuscular and subcutaneous injection as well as percutaneous air-driven administration (e.g., needleless injection). The vaccine may be given in a single dose or as multiple administrations. Where multiple administrations are given, they are preferably separated by at least one day, week, or month.

SVI Antibodies

Antibodies against SVI may be prepared using the isolated viral particles and/or SVI viral proteins provided by the instant invention. Isolated polyclonal antibodies as well as monoclonal antibodies may be made.

Isolated polyclonal antibodies against SVI proteins are preferably prepared by injecting a “SVI immunogen” (e.g., isolated SVI viral particles, SVI protein(s), SVI oligopeptides linked to a carrier, or SVI fusion proteins) in an immunogenic form into an animal, preferably a mammal such as a rodent (e.g., a mouse, rat or rabbit), a goat, a cow or a horse. Most commonly, the first injection of SVI immunogen is made as an oil/water emulsion complete adjuvant such as Freund's complete adjuvant, which contains a non-specific activator of the immune system to improve immune response to the injected immunogen. Later injections are typically made with incomplete adjuvant (e.g., in an emulsion w/o a non-specific immune stimulator). Alternately, the SVI immunogen can be introduced adsorbed to a solid substrate or as a simple solution. Serum is harvested and tested for the presence of specific antibody using any convenient assay, most typically a simple immunoassay such as an ELISA (enzyme-linked immunosorbent assay) using an SVI immunogen as the target and a species-specific anti-immunoglobin secondary antibody.

Monoclonal antibodies of this invention can be prepared by a number of different techniques. For hybridoma technology, the reader is referred generally to Harrow & Lane (1988), U.S. Pat. Nos. 4,491,632, 4,472,500, and 4,444,887, and Methods in Enzymology, 73B:3 (1981). Traditional monoclonal antibody technology involves the immortalization and cloning of an antibody-producing cell recovered from an animal, typically a mouse, that has been immunized as described in the preceding paragraph. The cell may be immortalized by, for example, fusion with a non-producing myeloma, infection with Epstein Barr Virus, or transformation with oncogenic DNA. The treated cells are cloned and cultured, and clones are selected that produce antibody of the desired specificity. Specificity testing is performed on culture supernatants by a number of techniques, such as using the immunizing antigen as the detecting reagent in an immunoassay. A supply of monoclonal antibody from the selected clone can then be purified from a large volume of culture supernatant, or from the ascites fluid of suitably prepared host animals injected with the clone.

Alternative methods for obtaining monoclonal antibodies involve contacting an immunocompetent cell or viral particle with a protein of the invention. In this context, “immunocompetent” means that the cell or particle has expressed or is capable of expressing an antibody specific for the antigen without further genetic rearrangement, and can be selected from a cell mixture by presentation of the antigen. Immunocompetent eukaryotic cells can be harvested from an immunized mammalian donor, or they can be harvested from an unimmunized donor and prestimulated in vitro by culturing in the presence of immunogen and immunostimulatory growth factors. Cells of the desired specificity can be selected by contacting with the immunogen under culture conditions that result in proliferation of specific clones but not non-specific clones. Immunocompetent phage may be constructed to express immunoglobulin variable region segments on their surface. See Marks et al., New Engl. J. Med. 335:730, 1996; International Patent Applications Nos. 94/13804, 92/01047, 90/02809; and McGuinness et al., Nature Biotechnol. 14:1149, 1996. Phage of the desired specificity may be selected, for example, by adherence to an SVI immunogen attached to a solid phase, and then amplified in E. coli.

Antibody can be purified from serum, cell supernatants, lysates, or ascites fluid by a combination of traditional biochemical separation techniques, such as ammonium sulfate precipitation, ion exchange chromatography on a weak anion exchange resin such as DEAE, hydroxyapatite chromatography, and gel filtration chromatography. Specific affinity techniques, such as affinity chromatography using an SVI immunogen as the affinity moiety may also be used, alone or in conjunction with traditional biochemical separation techniques to isolate antibodies of the invention.

Antibodies obtained are preferably screened or purified not only for their ability to react with SVI viral proteins, but also for a low cross-reactivity with potential cross-reacting substances also present in samples of diagnostic interest. Unwanted activity can be adsorbed out of polyclonal antisera, if necessary, using the cross-reacting substance or an antigen preparation from serum from an individual negative for SVI infection.

The epitope to which a particular antibody binds can be mapped by preparing fragments and testing the ability of the antibody to bind. For example, sequential peptides of 12 amino acids are prepared covering the entire sequence of the immunogen, and overlapping by 8 residues. The peptides can be prepared on a nylon membrane support by F-Moc chemistry, using a SPOTS™ kit from Genosys according to manufacturer's directions. Prepared membranes are then overlaid with the antibody, washed, and overlaid with β-galactose conjugated anti-human IgG. The test is developed by adding the substrate X-gal. Positive staining indicates an antigen fragment recognized by the antibody. The fragment can then be used to obtain other antibodies recognizing the epitope of interest. Two antibodies recognizing the same epitope will compete for binding in a standard immunoassay.

Antibodies of the invention may be used for the detection and/or identification of SVI virus, and may also be useful in isolation of viral particles and/or viral proteins.

Detection of SVI

The polynucleotides, proteins, and antibodies of the invention may be used in methods and kits for the detection of SVI viral infection and detection of SVI itself. Assays using the polynucleotides, proteins, and/or antibodies of the invention may be designed in a variety of formats, depending on the desired utility of the assay.

Polynucleotides of the invention may be used for detection of SVI virus genomic DNA. Detection of SVI genomic DNA in blood samples indicates that the sample is contaminated with SVI virus and that the source of the sample is infected with SVI. A wide variety of different assays for detection of nucleotides are known, although all such assays generally require a hybridization step where a primer or probe is hybridized to DNA in the sample.

Using determined portions of the isolated SVI genomic sequence as a basis, oligomers of approximately eight nucleotides or more can be prepared, either by excision or synthetically, which hybridize with the SVI genome. The natural or derived probes for SVI polynucleotides are a length that allows the detection of unique viral sequences by hybridization. Generally, probes are a minimum of six to eight nucleotides in length, sequences of at least ten to twelve nucleotides are preferred, and those of at least about 20 nucleotides may be most preferred. Depending on the desired utility of the assay (e.g., detection of all SVI viruses vs. detection of a single SVI virus type), the probe may be based on a region of SVI genomic sequence which is conserved among SVI viruses or highly divergent among SVI viruses. These probes can be prepared using routine, standard methods including automated oligonucleotide synthetic methods. A complement of any unique portion of the SVI genome will be satisfactory, although probes are preferably selected from regions which are divergent from known TT viruses. Generally, complete complementarity is desirable in probes, although it may be unnecessary as the length of the fragment is increased.

Normally, the test sample to be analyzed, such as blood or serum, is treated such as to extract the nucleic acids contained therein. The nucleic acid sample is typically adsorbed to a solid support (e.g., nitrocellulose) for the assay (with or without preliminary size separation such as by gel electrophoresis), although solution phase format assays such as the assay described in U.S. Pat. No. 4,868,105 may also be used.

Depending on the format of the assay and the detection system, the probes may or may not be directly labeled or otherwise modified to allow later detection by binding of a label. Suitable labels and methods for attaching labels to probes are known in the art, and include but are not limited to radioactive labels incorporated by nick translation or kinasing, modifications which allow later binding of a label, such as biotinylation, as well as fluorescent and chemiluminescent labels which may be directly bound to the probe or bound via a modification of the probe.

In the basic nucleic acid hybridization assay, single-stranded sample nucleic acid is contacted with the probe under hybridization and wash conditions of suitable stringency, and resulting duplexes are detected. Control of stringency is well known in the art, and depends on variables such as salt concentration, probe length, formamide concentration, temperature and the like. Preferably, hybridization and washing is performed under stringent conditions. Detection of bound probe is performed according to requirements of the label/detection system employed in the assay. For example, where the probe is radioactively labeled, probe binding is detected by autoradiography. Where the probe is modified to allow later binding of a label (e.g., by covalent linkage of biotin or digoxigenin to the probe or by addition of a polyA tail to the probe), a label linked to a modification binding moiety (e.g., streptavidin linked to a detectable enzyme such as alkaline phosphatase, green fluorescent protein or luciferase, or a fluorescent or other label bound to an anti-digoxigenin antibody). Detection of fluorescent probes is generally accomplished by fluorimeter, while luminescent labels may be detected using a luminometer or a photographic plate. Branched DNA technology and other methods which amplify signal from the assay may be employed (Urdea et al., 1989, Clin. Chem. 35(8):1571-1575; U.S. Pat. No. 5,849,481;).

Other assays employ probes as primers for amplification of SVI genomic DNA in the sample. Methods such as polymerase chain reaction, ligase chain reaction, Q-beta replicase, NASBA (Compton, 1991, Nature 350(6313):91-92),, etc., may be used to create large numbers of copies of a portion or all of the SVI genomic DNA present in a sample. Detection in such assays is normally by detection of an amplification product of an expected size, typically by gel electrophoresis and visualization of any bands present.

SVI virus may also be detected using antibodies of the invention to detect the presence of viral proteins in a sample. Any of the wide variety of immunoassay formats known in the art may be used in conjunction with antibodies of the invention for detection of SVI virus or viral proteins.

In its most basic form, an immunoassay for detection of SVI virus or SVI protein in a sample detects a complex of SVI protein(s) with an antibody of the invention. At least one antibody of the invention is required, although preferred immunoassay formats require at least two antibodies of the invention.

Many assay formats require that sample or the SVI proteins from the sample, be immobilized on a solid support. Linkage can be accomplished by a variety of means known in the art, most commonly adsorption to a protein binding surface (e.g., a polystyrene plate or nitrocellulose or PVA membrane) or binding to an antibody which is bound to the substrate. This second arrangement is used in “sandwich” immunoassays and is preferred for detection of SVI virus proteins.

After immobilization of the sample (or the SVI proteins in the sample) to the substrate, a detection antibody is contacted with the sample and the presence of the detection antibody is detected. The detection antibody may itself be detectable due to modification of the antibody with a dye or colored particles, it may be modified such that a detection reagent will bind to the detection antibody, or it may be modified with an enzyme which acts on a chromogenic substrate

The exact details of detection of the detection antibody will, of course, depend on the detection system utilized. Directly detectable detection antibodies may be detected by, for example, simple inspection, light microscopy or colorimetry (for antibodies modified with colored particles such as latex beads or colloidal metals), radiometry (for antibodies modified with a radioactive compound) or fluorimetry or epifluorescence microscopy (for antibodies labeled with fluorescent dyes). Detection antibodies that have been modified to include an enzyme are typically detected by incubating the assay in a solution containing a substrate that becomes detectable upon processing by the enzyme (e.g., substrates that change color or become fluorescent or luminescent after processing by the enzyme) and detecting any processed substrate using an appropriate method (e.g., colorimetry for chromogenic substrates, fluorimetry for fluorescent substrates, and the like). Other detection antibodies may be modified to allow for “indirect” detection, where a second reagent that binds to the modified detection antibody allows detection of bound detection antibody. The second reagent is modified such that it is detectable (either directly as with a dye or colored particle, indirectly as with an enzyme and detectable substrate).

### EXAMPLES

### Example 1

Isolation of SVI Virus DNA

DNA clones comprising SVI genomic DNA were isolated using a modification of the representation difference analysis (RDA) method described by Lititsyn et al. (1993, Science 259:946-951). This method utilizes a “driver” DNA source to enrich amplification of sequences unique to the “tester” DNA source.

Serum from a cryptogenic hepatitis patient designated H035 was used as the source of “tester” DNA. DNA was extracted by proteinase K digestion followed by phenol and chloroform extraction. DNA isolated from 100 μL H035 serum was digested to completion with 10 units of Sau3A I for three hours at 37° C. The enzyme was inactivated by incubation at 65° C. for 20 minutes.

Linkers R-Bgl-24 (5′-AGCACTCTCCAGCCTCTCACCGCA-3′) and R-Bgl-12 (5′-GATCTGCGGTGA-3′) were ligated to the digested DNA by mixing the digested DNA with 1 nmol of each oligo in T4 DNA ligase buffer (with ATP, from New England Biolabs), denaturing the mixture by incubation for two minutes at 55° C., annealing the linkers by gradually cooling the mixture to 10-15° C. over about an hour, then adding 800 units of T4 DNA ligase (New England Biolabs) and incubating overnight at 12-16° C.

Tester amplicons were prepared by amplifying a portion of the ligation product. A portion of the ligation product was mixed with PCR buffer, dNTPs, and an additional 250 pmol of R-Bgl-24 oligo and overlaid with mineral oil. The R-Bgl-12 oligo was ‘released’ by incubating the mixture for three minutes at 72° C. Overhangs were filled by adding 7.5 units of AMPLITAQ® Taq DNA polymerase (PE Biosystems) and incubating for a further five minutes at 72° C. Tester amplicons were created by running the mixture through 20 cycles of one minute at 95° C. and three minutes at 72° C. followed by a final extension step at 72° C. for ten minutes. The product was then extracted with phenol/chloroform and precipitated with sodium acetate and isopropanol. The precipitate was collected by centrifugation, air dried after removal of the supernatant, and resuspended in TE (tris-EDTA) buffer. The R-Bgl-24 linkers were removed by Sau3A I digestion essentially as above, followed by inactivation of the enzyme at 65° C. The digestion product was precipitated using sodium acetate and ethanol in the presence of glycogen, collected by centrifugation, air dried following removal of the supernatant, and resuspended in TE. The product was then separated on a 1% agarose gel run in 1× TAE, and the portion of the gel corresponding to 150-1500 nucleotides was cut out. Digested tester amplicons were purified from the gel using a QIAGEN® Qiaex II Gel Extraction kit according to the manufacturer's instructions. 2 μg of tester amplicon DNA was ligated with J-Bgl-24 and J-Bgl-12 linkers (5′-ACCGACGTCGACTATCCATGAACA-3′ and 5′-GATCTGTTCATG-3′, respectively), essentially as described for the R-Bgl linkers.

Driver amplicons were prepared from DNA extracted from serum pooled from 10 healthy blood donors essentially as described for tester amplicons, except that new linkers were not added after the second Sau3A I digestion.

Driver and tester amplicons were mixed at a 100:1 mass ratio, extracted with phenol/chloroform, and precipitated with sodium acetate and ethanol. The pellet was collected by centrifugation, air dried following removal of the supernatant, and resuspended in 4 μL of EE×3 buffer (30 mM EPPS, pH 8.0, 3 mM EDTA). The mixture was overlaid with mineral oil hybridized by denaturing for five minutes at 98° C., adding 1 μL of 5 M NaCl, incubating a further two minutes at 98° C., then 20 hours at 65° C.

The tester/driver hybridization mix was amplified under conditions which selectively amplify only double stranded tester DNA. A portion of the hybridization mix was amplified for 10 cycles essentially as was done for amplification of the J-Bgl ligated tester DNA, except that the extension cycles were performed at 70° C. The amplification product was collected, extracted with phenol/chloroform/isoamyl alcohol, then precipitated with sodium acetate and isopropanol. The precipitate was collected by centrifugation, air dried following removal of the supernatant, and resuspended in TE. Single stranded DNA was removed by digestion with mung bean nuclease (New England BioLabs) for 30 minutes at 30° C., followed by heat inactivation of the enzyme at 98° C. for five minutes. The digestion product was the re-amplified for 15 cycles in the presence of additional J-Bgl-24 oligonucleotide.

The product of the amplification was collected, extracted with phenol/chloroform/isoamyl alcohol, precipitated with sodium acetate and isopropanol, collected by centrifugation, washed with 70% ethanol, air dried following removal of the supernatant, and resuspended in TE to form the First Difference Product (DP1).

The Second Difference Product (DP2) was created by digesting DP1 with Sau3A I and substituting N-Bgl linkers (N-Bgl-12 and N-Bgl-24, 5′-GATCTTCCCTCG-3′ and 5′-AGGCAACTGTGCTATCCGAGGGAA-3′, respectively) for the J-Bgl linkers essentially as described for the switch from the R-Bgl linkers to the J-Bgl linkers, hybridizing the N-linker DP1 with driver amplicons at a 1:800 mass ratio, and amplifying/digesting/amplifying as described for DP1, except that extensions during the amplifications were carried out at 72° C.

The Third Difference Product (DP3) was created by digesting DP2 with Sau3A I and substituting J-Bgl linkers for the N-Bgl linkers, followed by followed by hybridization with driver amplicons at a 4×10⁵:1 driver:tester mass ratio, and amplification/digestion/amplification as for DP1.

After three rounds subtractive hybridization and selective amplification, distinct bands were seen after gel electrophoresis, as compared to the ‘smear’ patterns of the original tester amplicons. DNA was isolated from each band using a QIAGEN® Gel Extraction Kit (Cat. No. 28704) according to the manufacturer's instructions, then ligated into a TA plasmid (InVitrogen Cat. K2000-01). The resulting libraries of plasmids were then transformed into E. coli and plated. 30 colonies from each library were selected and sequenced using a PE Biosciences PCR sequencing kit according to the manufacturer's instructions. Sequences were compared against the GenBank database at the DNA and protein levels. Clones that showed no significant homology with the databases were classified as “unknowns.” Each “unknown” was tested for its presence in human genomic DNA by PCR using primers designed from each “unknown” sequence. Analysis was discontinued for any sequence present in human genomic DNA. One 314 nucleotide unknown, designated “clone 37” was selected for further characterization. The non-human origin of clone 37 was confirmed by Southern blot of human genomic DNA.

A library was created to isolate larger DNAs including the clone 37 sequence. DNA was isolated by proteinase K and phenol/chloroform extraction, cut with NcoI and BspH1, and ligated into a library vector. Genomic DNA from the prototypic SVI virus was isolated, as well as several variants, the sequences of which are shown in SEQ ID NO: 1 through SEQ ID NO: 5.

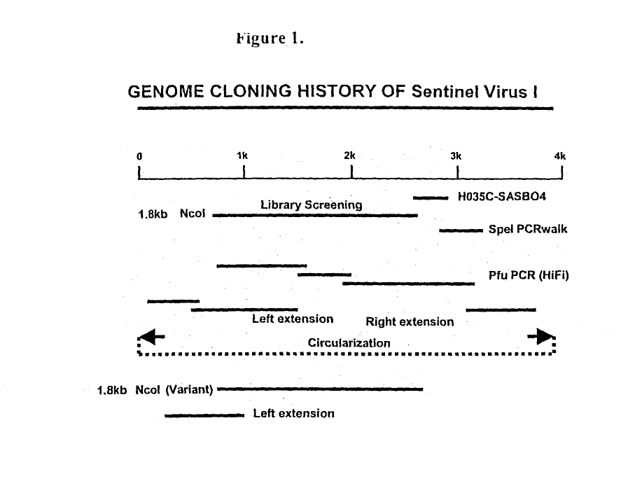

As shown in FIG. 1, higher fidelity sequence was obtained using higher titered starting material acquired from chimpanzees infected with the original SVI containing sample (see example 3 below). Primers homologous to the original sequence were designed and used with Pfu DNA polymerase to clone out fragments corresponding to the original sequence. Analysis of these sequences by comparison with the GenBank database at the DNA and protein levels demonstrated some homology to known circoviral sequences. The genomic sequence was extended to the right and to the left, by designing primers for PCR which paired regions in the known sequence with circovirus conserved regions. Finally, the genome was demonstrated to be circular and the sequence closing the circle determined by designing a primer pair in such a way that each primer went outward from the left and right ends. A fragment created by such a PCR reaction would be the result of a circular genome. Such a fragment was obtained and sequenced to complete the genomic sequence.

Computer analysis of both the high fidelity and variant sequences determined the presence of open reading frames in analogous positions and with limited homology to open reading frames found in the human circovirus isolates SANBAN and TUS01. There are two such open reading frames present in SVI and its variants, designated ORF1 (SEQ ID NO:6 through SEQ ID NO:10) and ORF2 (SEQ ID NO:11 and SEQ ID NO:12). The sequences listed herein as SEQ ID NO:6 through SEQ ID NO:10 respectively correspond to the ORF1 sequences of the polynucleotide sequences designated as SEQ ID NO:1 through SEQ ID NO:5. The peptide sequence herein designated as SEQ ID NO:11 details the ORF2 sequence of the polynucleotides designated as SEQ ID NO:1 and SEQ ID NO:2. Further, the peptide sequence herein designated as SEQ ID NO:12 details the ORF2 sequence of the polynucleotides designated as SEQ ID NO: 3, SEQ ID NO: 4 and SEQ ID NO: 5.

### Example 2

Physical Characterization of SVI Virus Particles

SVI positive serum was fractionated by density gradient ultracentrifugation to determine the buoyant density of SVI viral particles.

A 200 μL sample of SVI positive serum, spiked with HBV as a marker, was layered on the surface of a continuous sucrose density gradient (20-65% sucrose, w/w). The sample was centrifuged for 39,000 rpm for 15 hours at 6° C. in a Beckman SW41Ti rotor. Fractions (500 μL) were collected by pumping from the bottom of the tube via glass capillary tube attached to silicone tubing.

Each fraction was assayed for SVI using two rounds of PCR amplification. The first round used primers 37.2 and 37.3 (5′-CTCGACCTGGAAAGTCCAGTC-3′ and 5′-TGCAAAGCAACAGACATGGAC-3′, respectively), and the second round used primers 37.4 and 37.5 (5′-TTCCTTGCCCTTGGAGGTCTG-3′ and 5′-TAGACAGCGTCGCGACTTAC-3′, respectively). Fractions were also assayed for HBV using two rounds of PCR; the first round with primers HBV1 and HBV4 (5′-CATCTTCTTRTTGGTCTTCTGG-3′ and 5′-CAAGGCAGGATAGCCACATTGTG-3′, respectively), and primers HBV3 (5′-CCTATGGGAGTGGGCCTCAG-3′) and HBV4. SVI virus was found in fractions corresponding to 1.23-1.24 g/cm³.

SVI buoyant density was also measured in CsCl gradient. A 200 μL sample of SVI positive serum, spiked with HBV as a marker, was layered on the surface of a homogenous CsCl solution (density 1.308 g/cm³). The sample was centrifuged for 33,000 rpm for 70 hours 6° C. in a Beckman SW41Ti rotor. Fractions were collected and analyzed as described for the sucrose gradient experiment. SVI was found in fractions corresponding to 1.33-1.35 g/cm³.

Buoyant density was also assayed for samples treated with Tween. Buoyant density was unchanged.

### Example 3

Prevalence of SVI Infection

More than 1500 serum samples were assayed by PCR for presence of SVI. The samples were divided into: (a) “super normal” blood donors (normal blood values, no hepatitis virus markers, and not implicated in transfusion-related events for ≧5 blood donations); (b) “normal” blood donors (meeting blood donation criteria), from Italy and England; (c) “disqualified” blood donors (healthy individuals not eligible for blood donation under current rules); (d) “hepatitis” patients, separated into cryptogenic, acute, chronic HBV, chronic HCV, and chronic HBV and HCV or HBV and HDV; and (e) “transfusion” recipients, subdivided into thalassemia and hemophilia patients, including hemophiliacs receiving only recombinant clotting factors.

Samples were assayed by PCR amplification of DNA extracted from serum samples using primers 37.2, 37.3, 37.4, and 37.5 as described in Example 2. Results are shown in Table 1.

TABLE 1

| Group | Sample | Positive |
|---|---|---|
| Super Normal | 100 | 5(5%) |
| Normal | 253 | 21(12%) |
| England | 153 | 6(4%) |
| Italy | 100 | 15(15%) |
| Disqualified | 222 | 28(13%) |
| Hepatitis | 390 | 102(26%) |
| Cryptogenic | 99 | 26(26%) |
| Acute | 35 | 13(37%) |
| Chronic HBV | 79 | 27(34%) |
| Chronic HCV | 98 | 20(20%) |
| Chronic HBV/HCV/HDV | 79 | 16(20%) |
| Transfusion | 167 | 108(65%) |
| Thalassemia | 82 | 61(74%) |
| Hemophilia | 85 | 47(55%) |
| Recombinant Factors | 13 | 2(15%) |

The results show that SVI has a high prevalence in individuals positive for hepatitis and is very high in individuals receiving blood products.

### Example 4

Infection and Transmission of SVI in Chimpanzees

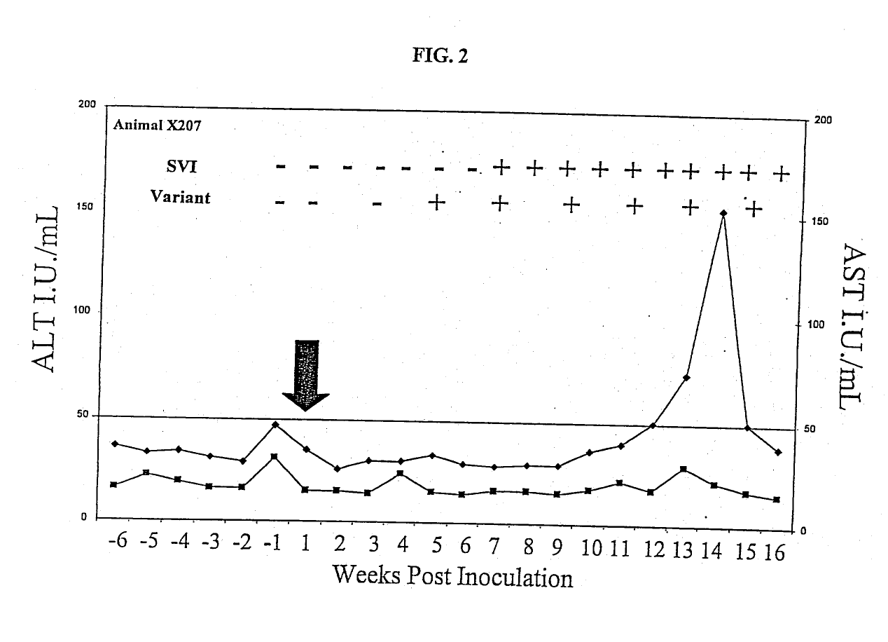

The infectiousness of SVI was assessed. A sample from a pool of sera including serum from patient H035 (i.e., SVI positive serum) was injected intravenously into a chimpanzee (designated X207). This animal tested negative for all known hepatitis viruses prior to administration of the SVI positive serum. Serum samples were obtained from this animal on a weekly basis and tested for the presence of the SVI virus by PCR using primers 37.2, 37.3, 37.4, and 37.5 as described in Example 2. Serum liver enzymes (ALT, AST) were also assayed, and certain samples were tested by PCR for SVI variant using primers which distinguish between prototypic SVI and the known variants of the virus (e.g., SEQ ID NO: 2 through SEQ ID NO: 5). The prototypic SVI was first detected seven weeks after inoculation, and variant 1 was detected 5 weeks after inoculation. As shown in FIG. 2, both prototypic SVI and SVI variant were detected in serum samples up to 16 weeks at the final time point assayed (16 and 15 weeks, respectively). These data indicate that SVI is an infectious agent that can infect and amplify in a non-human primate. However, it is unclear if the increase in ALT following inoculation with the SVI positive serum was due to SVI virus, as the serum pool was later found to be contaminated with HCV.

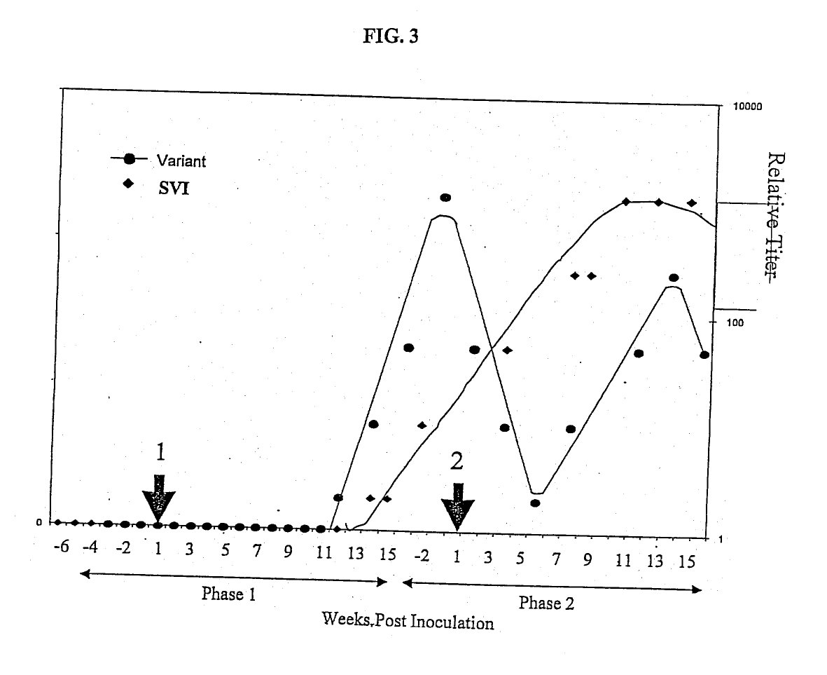

A second chimpanzee, designated X323, was inoculated with a pool of serum from five hepatitis patients which was believed to be free of SVI virus, although more extensive testing found that one of the donors included in the pool showed a low titer of SVI. This animal had also been previously tested negative for all known hepatitis viruses prior to inoculation. Serum samples were obtained from this animal weekly and tested for prototypic SVI and SVI variant by PCR using primers 37.2, 37.3, 37.4, and 37.5 as described in Example 2; serum liver enzymes (ALT, AST) were also assayed. No significant change in ALT or AST was observed, but as shown in FIG. 3, both the prototypic SVI and SVI variants were detected beginning at 12-14 weeks after the first inoculation.

17 weeks after the first inoculation, animal X323 was also inoculated with serum pooled from the week 8-14 samples from animal X207. PCR testing showed that SVI viremia persisted in animal X323 for at least 16 weeks after the second inoculation. In addition, prototypic SVI virus was also detected by PCR in DNA extracted from liver biopsies obtained from weeks 11 to 18 post inoculation, and in leukocytes from weeks 4 to 18 post inoculation.

In summary, we have demonstrated that SVI virus is able to infect and amplify in non-human primates, and SVI infection can be transmitted between non-human primates. The data also suggests that SVI may be able to establish chronic infections in non-human primates.

### Example 5

Transmission of SVI Virus in Humans

The transmissibility of SVI virus in humans was assessed. Pairs of pre- and post-transfusion serum from human recipients of blood transfusion, as well as the donor blood, were obtained Dr. Hitzler at Mainz. All samples were tested for SVI target sequences by PCR using primers 37.2, 37.3, 37.4, and 37.5 as described in Example 2.

Among the 206 sets of serum samples screened, eight transfusion recipients were identified who tested negative for SVI before transfusion and had received SVI positive blood. These eight patients were followed up by studies supported with clinical information and standard blood chemistry analysis beginning at two weeks post-transfusion, and subsequently at two-week intervals for at least two months. Among these eight transfusion recipients, six became SVI positive: one became SVI positive as early as 3-4 weeks post blood transfusion, four remained SVI positive at the time point of 15-16. weeks post transfusion and one was still SVI positive at the time point of 17-18 weeks post blood transfusion, suggesting that the SVI virus may establish chronic infections in humans. The data are summarized in Table 2. Note that SVI PCR assays were performed only at those time points noted by a “−” or a “+.”

TABLE 2

| | Weeks Post-Transfusion | | | | | | | | |
|---|---|---|---|---|---|---|---|---|---|
| Patient | 1/2 | 3/4 | 5/6 | 7/8 | 9/10 | 11/12 | 13/14 | 15/16 | 17/18 |
| KR11708 | | − | | | | | | | |
| KR11723 | | − | | | | | | | |
| KR11680 | | − | | | | + | | + | |
| KR11745 | | + | | | | | | | |
| RR13124 | | | | | | + | + | + | |
| KR13355 | | | | | + | + | | + | |
| KR13440 | | | | | | | + | + | + |
| KR16328 | | | | | + | + | + | | |

The patents, patent applications, and publications cited throughout the disclosure are incorporated herein by reference in their entirety.

The present invention has been detailed both by direct description and by example. Equivalents and modifications of the present invention will be apparent to those skilled in the art, and are encompassed within the scope of the invention.

We claim:

1. An isolated polyclonal antibody which binds to an SVI virus or a protein thereof, wherein said antibody does not bind to TTV strains SANBAN and TUS01 or proteins thereof.

2. A monoclonal antibody which binds to an SVI virus or a protein thereof, wherein said antibody does not bind to TTV strains SANBAN and TUS01 or proteins thereof.

3. A method for detecting SVI virus, comprising:
contacting a sample with an antibody which binds to SVI virus or a protein thereof, wherein said antibody does not bind to TTV strains SANBAN and TUS01 or proteins thereof; and
detecting complexes of said antibody and SVI virus or protein thereof.

4. An antibody that binds to an SVI virus wherein the genome of said virus contains any one of SEQ ID NO: 1, SEQ ID NO: 2, SEQ ID NO: 3, SEQ ID NO: 4, AND SEQ ID NO: 5.

5. The antibody of claim 4 wherein the antibody is a monoclonal antibody.

6. The antibody of claim 4 wherein the antibody is an isolated polyclonal antibody.

7. A method of detecting SVI virus, comprising:
contacting a sample with an antibody that binds to a virus or a protein thereof, wherein the genome of said virus contains any one of SEQ ID NO: 1, SEQ ID NO: 2, SEQ ID NO: 3, SEQ ID NO: 4, AND SEQ ID NO: 5; and
detecting complexes of said antibody and SVI virus or protein thereof.


SEQUENCE LISTING (25 sequences)

SEQ ID NO: 1 - DNA, 3847 bp, VIRUS
  misc_feature (221)..(221): This nucleotide is a T or C
agggttggcgtgtaacccggaagtcaatcctcccacatggcctgtcacgtgactgctacgtcacggccgccattttagttcacaaaatggcggacttccttcctcttttttaaaaattaaccgtcggcggcgctaccggcgcgcgccctgcgccccccccccctcgatgcgcgcggggcccccccccactgccgccccccggccccccccccgaagtacgncactaaccacgtgantcccacaggccaatcagagtctangtcgtgcacttcctgggcatggtctacgtcctaatataagtaagtgcacttccgaatggctgagttttccacgcccgtccgcagcggtagcaccacggaaggtgatccccgcgtcccgagggcgggtgccggaggtgagtttacacaccgcagtcaaggggcaattcgggctcgggactggccgggctatgggcaaggctcttagggtcatcgctcttaaaaatgttttttggcaggccttacagaaaaaagaaaagggcgctgctactgtctgagctgccagattcaaagaagaaaccacctggtgatatgacctggcgccccccagtacacaatgccgccggcatcgataggcagttctttgaagcctgctttcgatgccacagtagttgctgcggctgcggcaattttattaatcatcttaatgtactggctactcgctatggctttactggggggccggcgccgccaggtggtcctcggccggcgccccaagtgaggcccgcgcttcccgcgccggagcccgacgccccgggcgctgagccgctaccatggcgtggagctggtggtggcaacgatggaggcgccgcggctggaaacccaggcgccgccgctggagacgcctacgatggagaagacctcgacgccctgttcgccgccgtcgccgaagacgcagagtaaggagacggaggtgggggaaaagacgggggcgacgcagacggtacacccgtagacgaaagagacgcattaagcccaaaaactttaaaaacaaactagtactgactcagtggcacccctctgtagtgagacgctgctttatcaggggcatagtccccattctaatatgcgggcacacaaaatggaacagtaactacgccctacatagcgaggactacacagaagagggccgctaccctcacggagggtccctctcgaccactacgtggtctctaaaggtgctgtacgacgagtacctgaagcaccacaacttctggggctacccaaacacccagctagacctggccagatacaggggctgcagcttcaccttctacagacacaaaaagactgactttatagtatggtggaacagaaagcctccctttaaactcaacaagctgagctgcccgtccttccacccaggcatgctcatgcaacagaaacacaaggtgctcatccctagctttgacactaaacccgggggcagagctaaaattagagtcaaaataaagccccccactcttttagaggacaagtggtacactcagcaagacttgtgtccggtaaacctggcgcaagttgtggttactgcggctgactttcaacatccgttctgctcaccacaaacgaacactccaaccacaacgttccaagtgttgaaagacatttattacaacactatgagcatctctgagcccccagaggcctatactaatgttaataaaccctctgagagtaaattgtttagcaagtactcagaagaactagaaaaaatattatattcaacgggctcctactggaactcttttcactccatggagtaccttaatccaggcataacagatacgtacaacaataaaatatttaactctcagcccaactcactaatatcttggctgtcaacaaaaggcagcaacaactttcaaactggaaacagcacccagtttggccacaacagctacaacccaaatgatagtaacattaaatcagccaggagtgcatactggactgccctaaacaaacctaataaccttgctgttaacatagcacaggcaaaggcagagagattcgagtaccacctaggctggtattctcccatactactaagcaaccacagaagcaacttaaactttgccagggcctaccaagacgttacttacaaccctaactgtgacagaggtgtaaagaacaaaatatgggttcagcctctcactaaacccaccacagagttcgatgaaaagagatgcaagtgtgtggtanagaacctgcccctgtgggcggccttctactgctaccacaactttgtagaagaggagctaggcatttcttctgagatctacaacgcctgtatagtctgtgtacagtgcccttatacgtctccccccatgtatgacaagaaaaacccaaataaggggtacgtgttctatgacgccctgtttggaaacggaaagaccccagacggacgcggacaggtagacgtgttctggcagcagcggtggtacccccgcatggcctgccaagtgcaagtcatgcacgacattaccatgaccggaccgttctcctaccacgacgaactagtgagtacacaactgactgccaagtacaaatttgacttcatgtggggtggaaacatgatctcctcacagatcatcaagaacccctgcaaagacacagacatggacaccacctatcccagtagacagcgtcgcgacttacaaattgttgacccacactccatggggccctccttcgcgttccacacgtgggactacagacgcggactgtttggtaaagatgctatcgacagagtgtcaaaacaacaagacgatgctccagactatcctaacccatacaaaaagcctagatattttcccccaacagacagggcagaccttcaagggcaagaagaagactggactttccaggtcgagaagcgaggggtcgtcgtcagaagagagcgatcaagaagcccaagaagaggaggtactccagttccagccaggacagcagctccagcaactccacctgcagctcgcgcagcagcagcagatcgggcagcagctccgattcctgttccaacaaatgctcaaaacccaggccaatctgcacctaaacccatatacatacacccagctataaacagggtgttcatgtttgaaccccagggtcccaggcctataactaccagagaggggtgggaggacgagtacagcactgccggcatctggggcaggcccgtccgcaccttctacacagacacccccttctaccccgaggttcccaaaatgcctcctcagtttcatgtatctttcaaactcggctggaaataaaaatacaaggcctttcactgttcacttgtcggtgtctacctcttaaggtcactaagcactccgagcgccagcgaggagtgcgaccccttcccctggtgcaacgccctcggcggccgcgcgctacgccttcggctgcgcgcggcacctcggacccccgctcgtgctgacacgcttgcgcgtgtcagaccacttcgggctcgcgggggtcggaaattttgctaaacagactccgagttgccattggacactgtagccgtgaatcagtaacgaaagtgagtggggccagacttcgccataaggcctttatcttctcgccatttgtccgtcgggaggggtcgctccaaacgcggaccccgttttcagcccttccggactacaaaatgggccattttagtgacgtaacagctgccattttaagtaaggcggaagcagctccactatacaaaatggcggcggagcacttccggcttgcccaaaatggcggtcaacctcttccgggtcaaaggtgagcagctacgtcataagtcacgtggnggg

SEQ ID NO: 2 - DNA, 3844 bp, VIRUS
agggttggcgtgtaacccggaagtcaatcctcccacatggcctgtcacgtgactgctacgtcacggccgccattttagttcacaaaatggcggacttccttcctcttttttaaaaattaaccgtcggcggcgctaccggcgcgcgccctgcgccccccccccctcgatgcgcgcggggcccccccccactgccgccccccggccccccccccgaagtacgycactaaccacgtgaytcccacaggccaatcagagtctawgtcgtgcacttcctgggcatggtctacgtcctaatataagtaagtgcacttccgaatggctgagttttccacgcccgtccgcagcggtagcaccacggaaggtgatccccgcgtcccgagggcgggtgccggaggtgagtttacacaccgcagtcaaggggcaattcgggctcgggactggccgggctatgggcaaggctcttagggtcatcgctcttaaaaatgttttttggcaggccttacagaaaaaagaaaagggcgctgctactgtctgagctgccagattcaaagaagaaaccacctggtgatatgacctggcgccccccagtacacaatgccgccggcatcgataggcagttctttgaagcctgctttcgatgccacagtagttgctgcggctgcggcaattttattaatcatcttaatgtactggctactcgctatggctttactggggggccggcgccgccaggtggtcctcggccggcgccccaagtgaggcccgcgcttcccgcgccggagcccgacgccccgggcgctgagccgctaccatggcgtggagctggtggtggcaacgatggaggcgccgcggctggaaacccaggcgccgccgctggagacgcctacgatggagaagacctcgacgccctgttcgccgccgtcgccgaagacgcagagtaaggagacggaggtgggggaaaagacgggggcgacgcagacggtacacccgtagacgaaagagacgcattaagcccaaaaactttaaaaacaaactagtactgactcagtggcacccctctgtagtgagacgctgctttatcaggggcatagtccccattctaatatgcgggcacacaaaatggaacagtaactacgccctacatagcgaggactacacagaagagggccgctaccctcacggagggtccctctcgaccactacgtggtctctaaaggtgctgtacgacgagtacctgaagcaccacaacttctggggctacccaaacacccagctagacctggccagatacaggggctgcagcttcaccttctacagacacaaaaagactgactttatagtatggtggaacagaaagcctccctttaaactcaacaagctgagctgcccgtccttccacccaggcatgctcatgcaacagaaacacaaggtgctcatccctagctttgacactaaacccgggggcagagctaaaattagagtcaaaataaagccccccactcttttagaggacaagtggtacactcagcaagacttgtgtccggtaaacctggcgcaagttgtggttactgcggctgactttcaacatccgttctgctcaccacaaacgaacactccaaccacaaccttccaggtgttgaaagaaatgtattatgacactataggcatagtcgacacacaagaaacttacacctcagttaataataaatctacatctaacacatactctcattttcttaaggatttagaaaacaagttatactccacgggttcctactggaactcctttcacacagcagaatacttaaaccccaatatcacagacacttacaaccaaaaaatctttaattctcatacaacactaataaattggctgtcaactaaaggcagtgataagtttcaaactggtaacagcacccagtttggttacaacagctacaacccaaatgatagtaacattaaatcagccaggagtgcatactggactgccctaaacaaacctaataaccttgctgttaacatagcacaggcaaaggcagagagattcgagtaccacctaggctggtattctcccatactactaagcaaccacagaagcaacttaaactttgccagggcctaccaagacgttacttacaaccctaactgtgacagaggtgtaaagaacaaaatatgggttcagcctctcactaaacccaccacagagttcgatgaaaagagatgcaagtgtgtggtasagaacctgcccctgtgggcggccttctactgctaccacaactttgtagaagaggagctaggcatttcttctgagatctacaacgcctgtatagtctgtgtacagtgcccttatacgtctccccccatgtatgacaagaaaaacccaaataaggggtacgtgttctatgacgccctgtttggaaacggaaagaccccagacggacgcggacaggtagacgtgttctggcagcagcggtggtacccccgcatggcctgccaagtgcaagtcatgcacgacattaccatgaccggaccgttctcctaccacgacgaactagtgagtacacaactgactgccaagtacaaatttgacttcatgtggggtggaaacatgatctcctcacagatcatcaagaacccctgcaaagacacagacatggacaccacctatcccagtagacagcgtcgcgacttacaaattgttgacccacactccatggggccctccttcgcgttccacacgtgggactacagacgcggactgtttggtaaagatgctatcgacagagtgtcaaaacaacaagacgatgctccagactatcctaacccatacaaaaagcctagatattttcccccaacagacagggcagaccttcaagggcaagaagaagactggactttccaggtcgagaagcgaggggtcgtcgtcagaagagagcgatcaagaagcccaagaagaggaggtactccagttccagccaggacagcagctccagcaactccacctgcagctcgcgcagcagcagcagatcgggcagcagctccgattcctgttccaacaaatgctcaaaacccaggccaatctgcacctaaacccatatacatacacccagctataaacagggtgttcatgtttgaaccccagggtcccaggcctataactaccagagaggggtgggaggacgagtacagcactgccggcatctggggcaggcccgtccgcaccttctacacagacacccccttctaccccgaggttcccaaaatgcctcctcagtttcatgtatctttcaaactcggctggaaataaaaatacaaggcctttcactgttcacttgtcggtgtctacctcttaaggtcactaagcactccgagcgccagcgaggagtgcgaccccttcccctggtgcaacgccctcggcggccgcgcgctacgccttcggctgcgcgcggcacctcggacccccgctcgtgctgacacgcttgcgcgtgtcagaccacttcgggctcgcgggggtcggaaattttgctaaacagactccgagttgccattggacactgtagccgtgaatcagtaacgaaagtgagtggggccagacttcgccataaggcctttatcttctcgccatttgtccgtcgggaggggtcgctccaaacgcggaccccgttttcagcccttccggactacaaaatgggccattttagtgacgtaacagctgccattttaagtaaggcggaagcagctccactatacaaaatggcggcggagcacttccggcttgcccaaaatggcggtcaacctcttccgggtcaaaggtgagcagctacgtcataagtcacgtggwggg

SEQ ID NO: 3 - DNA, 2499 bp, VIRUS
cgccactaaccacgtgactcccacaggccaatcagagtctatgtcgtgcacttcctgggcatggtctacgtcctaatataagtaagtgcacttccgaatggctgagttttccacgcccgtccgcagcggtagcaccacggaaggtgatccccgcgtcccgagggcgggtgccggaggtgagtttacacaccgcagtcaaggggcaattcgggctcgggactggccgggctatgggcaaggctcttagggtcatcgctcttaaaaatgttttttggcaggccttacagaaaaaagaaaagggcgctgctactgtctgagctgccagattcaaagaagaaaccacctggtgatatgacctggcgccccccagtacacaatgccgccggcatcgataggcagttctttgaagcctgctttcgatgccacgctggttgctgtgggtgcggcagttttattaatcatcttaatgtattggctgctcgctatggctttactggggggccggcgccgccaggtggtcctcggccggtgccccaagtgaggcccgcgcttcccgcgccggagcccgaccccgaggcccacaaccgtgagccatggcgtggagctggtggtggcaacgatggagacggcgccgctggaaacccaggaggcgccgctggagacgcctacgatggagaagacctagacgcgctgttcgccgccgtcgccgaggacgcagagtaaggaggcggaggtgggcgcgtagacgggggcgacgcagacggtacgccaccagacgaaagagacgttttaggggtcgccgctttagaaagaaactagtactaactcagtggcaccccagtactatgagacgctgtttaataaggggcatagttccactggtcatatgcggccacaccagatggagctacaactacgcccttcacagcgaggactataccgaagagggtcgataccctcacggagggtccctcagcaccactacgtggtcccttaaggtcctctacgacgaatacatgaaacaccacaacttttggggctaccccaacaaccagctagacctggccaggtacaagggggccaaattcaccttttacagacacaaaaagactgactttattgtattctttaacagaaagcctccctttaaacttaacaaatacagctgcccttcctatcaccctggcatgcttatgcagcaaaaacacaaaatactaataccaagctacgacactaaacccaagggcaagccaaaaataactgttaaaataaagccccccactctcttagaggacaagtggtacacccagcaagacctgtgtaacgttaacctgttgcaacttgtggttactgcggctaactttcaacatccgttctgctcaccacaaacgaacactccaaccacaaccttccaggtgttgaaagacatatattatgacactatgagcatatctgaccccacagacaactacaccagtgttaatgatttaaataaaacagacacttttagtgcttattcaaaaaaattagaagagattctatataaacgggcgtcctactggaactcgttccacgccacagagtacgtaaacccaaacattatttacaaaaatggacaaaaactttttaacaacaacagtgatttaataacttggatgacacaaactaacgaaaatggttttttaactaaaaacaacactgcatttggtaacaacagttataggccaaacttagacaaaattaaagaagctagacataaatactgggaagcccttacaggcaacaacgacctagccactagcataggccaggccaaagcagaacggttcgagtaccacctaggctggtactcccctatatttcttagcagacacagaagcaacatgaactttgccagagcctaccaagacgttacatacaaccccaactgtgacaggggagttaaaaacagagtgtggattcagcctctcactaaacccactacagagtttgacgagaaaaggtgcaagtgcgtcgtagaaaacctgcctctatggagcgccctgtactgctaccaagactttgtagaacaagagctaggtacatcttcggagatattaaattcatgcttattagtagtgcagtgcccctacacctttccacccatgtatgacaaaaaactacctaacaagggatatgtattctacgactccctatttggagacggcaaaatgtctgacggacgcggacaggtaaacattttctggcaacagcgatggtaccctcgcttagccactcaaatacaagttatgcatgacattaccattaccggccccttttcctaccgagacgagctagtagccacacaactgactgcaaagtacacctttgactttatgtggggcggtaatatgatctccacacagatcatcaagaacccctgcaaagacagtggactggaacccgcctaccccggtagactccgtcgcgacttacaaattgttgacccatactccatgg

SEQ ID NO: 4 - DNA, 2499 bp, VIRUS
cgccactaaccacgtgactcccacaggccaatcagagtctatgtcgtgcacttcctgggcatggtctacgtcctaatataagtaagtgcacttccgaatggctgagttttccacgcccgtccgcagcggtagcaccacggaaggtgatccccgcgtcccgagggcgggtgccggaggtgagtttacacaccgcagtcaaggggcaattcgggctcgggactggccgggctatgggcaaggctcttagggtcatcgctcttaaaaatgttttttggcaggccttacagaaaaaagaaaagggcgctgctactgtctgagctgccagattcaaagaagaaaccacctggtgatatgacctggcgccccccagtacacaatgccgccggcatcgataggcagttctttgaagcctgctttcgatgccacgctggttgctgtgggtgcggcagttttattaatcatcttaatgtattggctgctcgctatggctttactggggggccggcgccgccaggtggtcctcggccggtgccccaagtgaggcccgcgcttcccgcgccggagcccgaccccgaggcccacaaccgtgagccatggcgtggagctggtggtggcaacgatggagacggcgccgctggaaacccaggaggcgccgctggagacgcctacgatggagaagacctagacgcgctgttcgccgccgtcgccgaggacgcagagtaaggaggcggaggtgggcgcgtagacgggggcgacgcagacggtacgccaccagacgaaagagacgttttaggggtcgccgctttagaaagaaactagtactaactcagtggcaccccagtactatgagacgctgtttaataaggggcatagttccactggtcatatgcggccacaccagatggagctacaactacgcccttcacagcgaggactataccgaagagggtcgataccctcacggagggtccctcagcaccactacgtggtcccttaaggtcctctacgacgaatacatgaaacaccacaacttttggggctaccccaacaaccagctagacctggccaggtacaagggggccagattcaccttttacagacacaaaaagactgactttattgtattctttaacagaaagcctccctttaaacttaacaaatacagctgcccttcctatcaccctggcatgcttatgcagcaaaaacacaaaatactaataccaagctacgacactaaacccaagggcaagccaaaaataactgttaaagtaaagccccccactctcttagaggacaagtggtacacccagcaagacctgtgtaacgttaacctgttgcaacttgtggttactgcggctaactttcaacatccgttctgctcaccacaaacgaacactccaaccacaaccttccaggtgttgaaagacatatattatgacactatgagcatatctgaccccacagacaactacaccagtgttaataatttaaatgaagcaaacagttttagtgtttattcacaaaaattagaaaacattctatatgaacgggcgtcctactggaactcgttccacgccacagagtacgtaaacccaaacattatttacaaaaatggagaaaaactttttaaaagcaatactaatttaatgacttggatgacacaagctgagggcaaaggttttttaactaaaaacaacactgcatttggtaacaacagttataagccaaactcagaaaaaattaaagaagctagaaaaatatactgggacgcccttacaggcagcaacgacctagccactcacataggccaggccagagcagaacggttcgagtaccacctaggctggtactcccctatatttcttagcagacacagaagcaacatgaactttgccagagcctaccaagacgttacatacaaccccaactgtgacaggggagttaaaaacagagtgtggattcagcctctcactaaacccactacagagtttgacgagaaaaggtgcaagtgcgtggtagaaaacctgcctctatggagcgccctgtactgctaccaagactttgtagaacaagagctaggtacatcttcggagatattaaattcatgcttattagtagtgcagtgcccctacacctttccacccatgtatgacaaaaaactacctaacaagggatatgtattctacgactccctatttggagacggcaaaatgtctgacggacgcggacaggtagacattttctggcaacagcgatggtaccctcgcttagccactcaaatacaagttatgcatgacattaccattaccggccccttttcctaccgagacgagctagtagccacacaactgactgcaaagtacacctttgactttatgtggggcggtaatatgatctccacacagatcatcaagaacccctgcaaagacagtggactggaacccgcctaccccggtagactccgtcgcgacttacaaattgttgacccatactccatgg

SEQ ID NO: 5 - DNA, 2496 bp, VIRUS
cgccactaaccacgtgactcccacaggccaatcagagtctatgtcgtgcacttcctgggcatggtctacgtcctaatataagtaagtgcacttccgaatggctgagttttccacgcccgtccgcagcggtagcaccacggaaggtgatccccgcgtcccgagggcgggtgccggaggtgagtttacacaccgcagtcaaggggcaattcgggctcgggactggccgggctatgggcaaggctcttagggtcatcgctcttaaaaatgttttttggcaggccttacagaaaaaagaaaagggcgctgctactgtctgagctgccagattcaaagaagaaaccacctggtgatatgacctggcgccccccagtacacaatgccgccggcatcgataggcagttctttgaagcctgctttcgatgccacgctggttgctgtgggtgcggcagttttattaatcatcttaatgtattggctgctcgctatggctttactggggggccggcgccgccaggtggtcctcggccggtgccccaagtgaggcccgcgcttcccgcgccggagcccgaccccgaggcccacaaccgtgagccatgacgtggagctggtggtggcaacgatggagacggcgccgctggaaacccaggaggcgccgctggagacgcctacgatggagaagacctagacgcgctgttcgccgccgtcgccgaggacgcagagtaaggaggcggaggtgggcgcgtagacgggggcgacgcagacggtacgccaccagacgaaagagacgttttaggggtcgccgctttagaaagaaactagtactaactcagtggcaccccagtactatgagacgctgtttaataaggggcatagttccactggtcatatgcggccacaccagatggagctacaactacgcccttcacagcgaggactataccgaagagggtcgataccctcacggagggtccctcagcaccactacgtggtcccttaaggtcctctacgacgaatacatgaaacaccacaacttttggggctaccccaacaaccagctagacctggccaggtacaagggggccaaattcaccttttacagacacaaaaagactgactttattgtattctttaacagaaagcctccctttaaacttaacaaatacagctgcccttcctatcaccctggcatgcttatgcagcaaaaacacaaaatactaataccaagctacgacactaaacccaagggcaagccaaaaataactgttaaaataaagccccccactctcttagaggacaagtggtacacccagcaagacctgtgtaacgttaacctgttgcaacttgtggttactgcggctaactttcaacatccgttctgctcaccacaaacgaacactccaaccacaaccttccaggtgttgaaagacatatattatgacactatgagcatatctgaccccacagacaactacaccagtgttaataatttaaataaaacagacagttttactgcttattcacaaaaattagaagagattctatatacacgggcgtcctactggaactcgttccacgccacagagtacgtaaacccaaacattatttacaaaaatggacaaaaactttttgacaacagtactttaacaacttggatgacacaaactaacgatgttggttttttaactaaaaacaacactgcatttggtaacaacagttataggccaaacgcagagaaaattaaacaagctagacagaaatactgggaagcccttacaggcaacaacgacctagccactagcataggccaggccaaagcagaacggttcgagtaccacctaggctggtactcccctatatttcttagcagacacagaagcaacatgaactttgccagagcctaccaagacgttacatacaaccccaactgtgacaggggagttaaaaacagagtgtggattcagcctctcactaaacccactacagagtttgacgagaaaaggtgcaagtgcgtcgtagaaaacctgcctctatggagcgccctgtactgctaccaagactttgtagaacaagagctaggtacatcttcggagatattaaattcatgcttattagtagtgcagtgcccctacacctttccacccatgtatgacaaaaaactacctaacaagggatatgtattctacgactccctatttggagacggcaaaatgtctgacggacgcggacaggtagacattttctggcaacagcgatggtaccctcgcttagccactcaaatacaagttatgcatgacattaccattaccggccccttttcctaccgagacgagctagtagccacacaactgactgcaaagtacacctttgactttatgtggggcggtaatatgatctccacacagatcatcaagaacccctgcaaagacagtggactggaacccgcctaccccggtagactccgtcgcgacttacaaattgttgacccatactccatgg

SEQ ID NO: 6 - protein, 768 aa, VIRUS
MAWSWWWQRWRRRGWKPRRRRWRRLRWRRPRRPVRRRRRRRRVRRRRWGKRRGRRRRYTRRRKRRIKPKNFKNKLVLTQWHPSVVRRCFIRGIVPILICGHTKWNSNYALHSEDYTEEGRYPHGGSLSTTTWSLKVLYDEYLKHHNFWGYPNTQLDLARYRGCSFTFYRHKKTDFIVWWNRKPPFKLNKLSCPSFHPGMLMQQKHKVLIPSFDTKPGGRAKIRVKIKPPTLLEDKWYTQQDLCPVNLAQVVVTAADFQHPFCSPQTNTPTTTFQVLKDIYYNTMSISEPPEAYTNVNKPSESKLFSKYSEELEKILYSTGSYWNSFHSMEYLNPGITDTYNNKIFNSQPNSLISWLSTKGSNNFQTGNSTQFGHNSYNPNDSNIKSARSAYWTALNKPNNLAVNIAQAKAERFEYHLGWYSPILLSNHRSNLNFARAYQDVTYNPNCDRGVKNKIWVQPLTKPTTEFDEKRCKCVVZNLPLWAAFYCYHNFVEEELGISSEIYNACIVCVQCPYTSPPMYDKKNPNKGYVFYDALFGNGKTPDGRGQVDVFWQQRWYPRMACQVQVMHDITMTGPFSYHDELVSTQLTAKYKFDFMWGGNMISSQIIKNPCKDTDMDTTYPSRQRRDLQIVDPHSMGPSFAFHTWDYRRGLFGKDAIDRVSKQQDDAPDYPNPYKKPRYFPPTDRADLQGQEEDWTFQVESEGSSSEESDQEAQEEEVLQFQPGQQLQQLHLQLAQQQQIGQQLRFLFQQMLKTQANLHLNPYTYTQL

SEQ ID NO: 7 - protein, 831 aa, VIRUS
  MISC_FEATURE (476)..(476): Unknown amino acid
MAWSWWWQRWRRRGWKPRRRRWRRLRWRRPRRPVRRRRRRRRVRRRRWGKRRGRRRRYTRRRKRRIKPKNFKNKLVLTQWHPSVVRRCFIRGIVPILICGHTKWNSNYALHSEDYTEEGRYPHGGSLSTTTWSLKVLYDEYLKHHNFWGYPNTQLDLARYRGCSFTFYRHKKTDFIVWWNRKPPFKLNKLSCPSFHPGMLMQQKHKVLIPSFDTKPGGRAKIRVKIKPPTLLEDKWYTQQDLCPVNLAQVVVTAADFQHPFCSPQTNTPTTTFQVLKEMYYDTIGIVDTQETYTSVNNKSTSNTYSHFLKDLENKLYSTGSYWNSFHTAEYLNPNITDTYNQKIFNSHTTLINWLSTKGSDKFQTGNSTQFGYNSYNPNDSNIKSARSAYWTALNKPNNLAVNIAQAKAERFEYHLGWYSPILLSNHRSNLNFARAYQDVTYNPNCDRGVKNKIWVQPLTKPTTEFDEKRCKCVVXNLPLWAAFYCYHNFVEEELGISSEIYNACIVCVQCPYTSPPMYDKKNPNKGYVFYDALFGNGKTPDGRGQVDVFWQQRWYPRMACQVQVMHDITMTGPFSYHDELVSTQLTAKYKFDFMWGGNMISSQIIKNPCKDTDMDTTYPSRQRRDLQIVDPHSMGPSFAFHTWDYRRGLFGKDAIDRVSKQQDDAPDYPNPYKKPRYFPPTDRADLQGQEEDWTFQVEKRGVVVRRERSRSPRRGGTPVPARTAAPATPPAARAAAADRAAAPIPVPTNAQNPGQSAPKPIYIHPAINRVFMFEPQGPRPITTREGWEDEYSTAGIWGRPVRTFYTDTPFYPEVPKMPPQFHVSFKLGWK

SEQ ID NO: 8 - protein, 635 aa, VIRUS
MAWSWWWQRWRRRRWKPRRRRWRRLRWRRPRRAVRRRRRGRRVRRRRWARRRGRRRRYATRRKRRFRGRRFRKKLVLTQWHPSTMRRCLIRGIVPLVICGHTRWSYNYALHSEDYTEEGRYPHGGSLSTTTWSLKVLYDEYMKHHNFWGYPNNQLDLARYKGAKFTFYRHKKTDFIVFFNRKPPFKLNKYSCPSYHPGMLMQQKHKILIPSYDTKPKGKPKITVKIKPPTLLEDKWYTQQDLCNVNLLQLVVTAANFQHPFCSPQTNTPTTTFQVLKDIYYDTMSISDPTDNYTSVNDLNKTDTFSAYSKKLEEILYKRASYWNSFHATEYVNPNIIYKNGQKLFNNNSDLITWMTQTNENGFLTKNNTAFGNNSYRPNLDKIKEARHKYWEALTGNNDLATSIGQAKAERFEYHLGWYSPIFLSRHRSNMNFARAYQDVTYNPNCDRGVKNRVWIQPLTKPTTEFDEKRCKCVVENLPLWSALYCYQDFVEQELGTSSEILNSCLLVVQCPYTFPPMYDKKLPNKGYVFYDSLFGDGKMSDGRGQVNIFWQQRWYPRLATQIQVMHDITITGPFSYRDELVATQLTAKYTFDFMWGGNMISTQIIKNPCKDSGLEPAYPGRLRRDLQIVDPYSM

SEQ ID NO: 9 - protein, 635 aa, VIRUS
MAWSWWWQRWRRRRWKPRRRRWRRLRWRRPRRAVRRRRRGRRVRRRRWARRRGRRRRYATRRKRRFRGRRFRKKLVLTQWHPSTMRRCLIRGIVPLVICGHTRWSYNYALHSEDYTEEGRYPHGGSLSTTTWSLKVLYDEYMKHHNFWGYPNNQLDLARYKGARFTFYRHKKTDFIVFFNRKPPFKLNKYSCPSYHPGMLMQQKHKILIPSYDTKPKGKPKITVKVKPPTLLEDKWYTQQDLCNVNLLQLVVTAANFQHPFCSPQTNTPTTTFQVLKDIYYDTMSISDPTDNYTSVNNLNEANSFSVYSQKLENILYERASYWNSFHATEYVNPNIIYKNGEKLFKSNTNLMTWMTQAEGKGFLTKNNTAFGNNSYKPNSEKIKEARKIYWDALTGSNDLATHIGQARAERFEYHLGWYSPIFLSRHRSNMNFARAYQDVTYNPNCDRGVKNRVWIQPLTKPTTEFDEKRCKCVVENLPLWSALYCYQDFVEQELGTSSEILNSCLLVVQCPYTFPPMYDKKLPNKGYVFYDSLFGDGKMSDGRGQVDIFWQQRWYPRLATQIQVMHDITITGPFSYRDELVATQLTAKYTFDFMWGGNMISTQIIKNPCKDSGLEPAYPGRLRRDLQIVDPYSM

SEQ ID NO: 10 - protein, 634 aa, Virus
MTWSWWWQRWRRRRWKPRRRRWRRLRWRRPRRAVRRRRRGRRVRRRRWARRRGRRRRYATRRKRRFRGRRFRKKLVLTQWHPSTMRRCLIRGIVPLVICGHTRWSYNYALHSEDYTEEGRYPHGGSLSTTTWSLKVLYDEYMKHHNFWGYPNNQLDLARYKGAKFTFYRHKKTDFIVFFNRKPPFKLNKYSCPSYHPGMLMQQKHKILIPSYDTKPKGKPKITVKIKPPTLLEDKWYTQQDLCNVNLLQLVVTAANFQHPFCSPQTNTPTTTFQVLKDIYYDTMSISDPTDNYTSVNNLNKTDSFTAYSQKLEEILYTRASYWNSFHATEYVNPNIIYKNGQKLFDNSTLTTWMTQTNDVGFLTKNNTAFGNNSYRPNAEKIKQARQKYWEALTGNNDLATSIGQAKAERFEYHLGWYSPIFLSRHRSNMNFARAYQDVTYNPNCDRGVKNRVWIQPLTKPTTEFDEKRCKCVVENLPLWSALYCYQDFVEQELGTSSEILNSCLLVVQCPYTFPPMYDKKLPNKGYVFYDSLFGDGKMSDGRGQVDIFWQQRWYPRLATQIQVMHDITITGPFSYRDELVATQLTAKYTFDFMWGGNMISTQIIKNPCKDSGLEPAYPGRLRRDLQIVDPYSM

SEQ ID NO: 11 - protein, 152 aa, VIRUS
MFFGRPYRKKKRALLLSELPDSKKKPPGDMTWRPPVHNAAGIDRQFFEACFRCHAGCCGCGSFINHLNVLAARYGFTGGPAPPGGPRPVPQVRPALPAPEPDPEAHNREPWRGAGGGNDGGAAAGNPGAAAGDAYDGEDLDALFAAVAEDAE

SEQ ID NO: 12 - protein, 110 aa, VIRUS
MFFGRPYRKKKRALLLSELPDSKKKPPGDMTWRPPVHNAAGIDRQFFEACFRCHAGCCGCGSFINHLNVLAARYGFTGGPAPPGGPRPVPQVRPALPAPEPDPEAHNREP

SEQ ID NO: 13 - DNA, 24 bp, Artificial Sequence
  Synthetic construct - linker sequence
agcactctccagcctctcaccgca

SEQ ID NO: 14 - DNA, 12 bp, Artificial Sequence
  Synthetic construct - linker sequence
gatctgcggtga

SEQ ID NO: 15 - DNA, 24 bp, Artificial Sequence
  Synthetic construct - linker sequence
accgacgtcgactatccatgaaca

SEQ ID NO: 16 - DNA, 12 bp, Artificial Sequence
  Synthetic construct - linker sequence
gatctgttcatg

SEQ ID NO: 17 - DNA, 12 bp, Artificial Sequence
  Synthetic construct - linker sequence
gatcttccctcg

SEQ ID NO: 18 - DNA, 24 bp, Artificial Sequence
  Synthetic construct - linker sequence
aggcaactgtgctatccgagggaa

SEQ ID NO: 19 - DNA, 21 bp, Artificial Sequence
  Synthetic construct - linker sequence
ctcgacctggaaagtccagtc

SEQ ID NO: 20 - DNA, 21 bp, Artificial Sequence
  Synthetic construct - linker sequence
tgcaaagcaacagacatggac

SEQ ID NO: 21 - DNA, 21 bp, Artificial Sequence
  Synthetic construct - linker sequence
ttccttgcccttggaggtctg

SEQ ID NO: 22 - DNA, 20 bp, Artificial Sequence
  Synthetic construct - linker sequence
tagacagcgtcgcgacttac

SEQ ID NO: 23 - DNA, 22 bp, Artificial Sequence
  Synthetic construct - linker sequence
catcttcttrttggtcttctgg

SEQ ID NO: 24 - DNA, 23 bp, Artificial Sequence
  Synthetic construct - linker sequence
caaggcaggatagccacattgtg

SEQ ID NO: 25 - DNA, 20 bp, Artificial Sequence
  Synthetic construct - linker sequence
cctatgggagtgggcctcag